(19) United States
(12) Patent Application Publication
(10) Pub. No.: US 2022/0315574 A1
(43) Pub. Date: Oct. 6, 2022

(54) Beta-Arrestin-Modulating Compounds and Methods of Using Same

(71) Applicant: Duke University, Durham, NC (US)

(72) Inventors: Robert Lefkowitz, Durham, NC (US); Alem KAHSAI, Durham, NC (US)

(21) Appl. No.: 17/630,667

(22) PCT Filed: Jul. 31, 2020
(86) PCT No.: PCT/US20/44403

§ 371 Date: Jan. 27, 2022

Related U.S. Application Data
(60) Provisional application No. 62/885,514, filed on Aug. 12, 2019.
(60) Provisional application No. 62/881,468, filed on Aug. 1, 2019.

(51) Int. Cl. C07D 417/12; C07D 217/20; C07D 307/80; C07D 493/14

(52) U.S. Cl. CPC C07D 417/12; C07D 217/20; C07D 307/80; C07D 493/14

(57) ABSTRACT

The present disclosure provides novel β-arrestin-modulating compounds and methods of making and using same.

### CROSS-RELATION TO OTHER APPLICATIONS

This application claims benefit of U.S. Provisional Patent Application No. 62/881,486, filed Aug. 1, 2019, and U.S. Provisional Patent Application No. 62/885,514, filed Aug. 12, 2019, and the contents of both application are herein incorporated in their entirety by reference.

### FEDERAL FUNDING LEGEND

This invention was made with Government support under Federal Grant nos. R01-HLBI and 3R01HL16037-45S1 awarded by the NIH/NHLBI. The Federal Government has certain rights to this invention.

### BACKGROUND

G protein coupled receptors (GPCRs), also known as seven transmembrane receptors (7TMRs), constitute the largest family of cell surface receptors and are targets for nearly one-third of marketed drugs (Hauser, A. S., et al., Trends in GPCR drug discovery: new agents, targets and indications. Nat Rev Drug Discov 16, 829-842, doi:10.1038/nrd.2017.178 (2017); Lefkowitz, R. J. A brief history of G-protein coupled receptors (Nobel Lecture). Angew Chem Int Ed Engl 52, 6366-6378, doi:10.1002/anie.201301924 (2013)). Agonist-stimulated GPCRs undergo conformational changes that promote association with and activation of heterotrimeric G proteins (Rasmussen, S. G. et al. Crystal structure of the beta2 adrenergic receptor-Gs protein complex. Nature 477, 549-555, doi:10.1038/nature10361 (2011); Oldham, W. M. & Hamm, H. E. Heterotrimeric G protein activation by G-protein-coupled receptors. Nat Rev Mol Cell Biol 9, 60-71, doi:10.1038/nrm2299 (2008)). This, in turn, modulates downstream effectors (e.g., enzymes that generate second messengers). Upon activation, GPCRs are rapidly desensitized through a two-step process (Luttrell, L. M. & Lefkowitz, R. J. The role of beta-arrestins in the termination and transduction of G-protein-coupled receptor signals. J Cell Sci 115, 455-465 (2002); Gurevich, V. V. & Gurevich, E. V. The structural basis of arrestin-mediated regulation of G-protein-coupled receptors. Pharmacol Ther 110, 465-502, doi:10.1016/j.pharmthera.2005.09.008 (2006); Ferguson, S. S. Evolving concepts in G protein-coupled receptor endocytosis: the role in receptor desensitization and signaling. Pharmacol Rev 53, 1-24 (2001); and Lefkowitz, R. J., et al., Dancing with different partners: protein kinase a phosphorylation of seven membrane-spanning receptors regulates their G protein-coupling specificity. Mol Pharmacol 62, 971-974, doi:10.1124/mol.62.5.971 (2002)): first, the receptor is phosphorylated by GPCR kinases (GRKs), primarily at multiple sites on the cytoplasmic carboxyl-terminal tail, followed by binding of arrestin to the phosphorylated receptor (Shukla, A. K. et al. Structure of active beta-arrestin-1 bound to a G-protein-coupled receptor phosphopeptide. Nature 497, 137-141, doi:10.1038/nature12120 (2013)). This binding event uncouples G proteins from the receptor and targets the receptor for internalization via clathrin-coated pits (Krupnick, J. G., et al., Arrestin/clathrin interaction. Localization of the clathrin binding domain of nonvisual arrestins to the carboxy terminus. J Biol Chem 272, 15011-15016, doi:10.1074/jbc.272.23.15011 (1997); Laporte, S. A. et al. The beta2-adrenergic receptor/betaarrestin complex recruits the clathrin adaptor AP-2 during endocytosis. Proc Natl Acad Sci USA 96, 3712-3717, doi:10.1073/pnas.96.7.3712 (1999)).

The arrestin family comprises two visual arrestins (arrestin-1 and arrestin-4) and two ubiquitously expressed non-visual forms (arrestin-2 and arrestin-3), generally referred to as β-arrestin-1 (βarr1) and β-arrestin-2 (βarr2), respectively. Beyond the canonical function as G protein signal terminators, non-visual arrestins have been identified as signal transduction units that promote pathways independent of or in concert with G proteins (Lefkowitz, R. J. & Shenoy, S. K. Transduction of receptor signals by beta-arrestins. Science 308, 512-517, doi:10.1126/science.1109237 (2005); Lefkowitz, R. J. Arrestins come of age: a personal historical perspective. Prog Mol Biol Transl Sci 118, 3-18, doi:10.1016/B978-0-12-394440-5.00001-2 (2013); Gurevich, V. V., et al., Arrestins as multi-functional signaling adaptors. Handb Exp Pharmacol, 15-37, doi:10.1007/978-3-540-72843-6_2 (2008); and Thomsen, A. R. B. et al. GPCR-G Protein-beta-Arrestin Super-Complex Mediates Sustained G Protein Signaling. Cell 166, 907-919, doi:10.1016/j.ce11.2016.07.004 (2016)). βarrs act as scaffolds and facilitate interactions with signaling mediators, such as the extracellular signal-regulated kinases 1 and 2 (ERK1/2), p38, and c-Jun N-terminal kinases (JNK-3) (Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); Luttrell, L. M. et al. Manifold roles of beta-arrestins in GPCR signaling elucidated with siRNA and CRISPR/Cas9. Sci Signal 11, doi:10.1126/scisignal.aat7650 (2018); and Luttrell, L. M. et al. Activation and targeting of extracellular signal-regulated kinases by beta-arrestin scaffolds. Proc Natl Acad Sci USA 98, 2449-2454, doi:10.1073/pnas.041604898 (2001)). Dysregulation of βarr function is linked to the etiology of inflammatory, metabolic, cardiovascular, neurologic, and oncogenic diseases (Walker, J. K. et al. Beta-arrestin-2 regulates the development of allergic asthma. J Clin Invest 112, 566-574, doi:10.1172/JCI17265 (2003); Ge, L., et al., A beta-arrestin-dependent scaffold is associated with prolonged MAPK activation in pseudopodia during protease-activated receptor-2-induced chemotaxis. J Biol Chem 278, 34418-34426, doi:10.1074/jbc.M300573200 (2003); Jiang, D., et al., beta-Arrestins in the immune system. Prog Mol Blot Transl Sci 118, 359-393, doi:10.1016/B978-0-12-394440-5.00014-0 (2013); Thathiah, A. et al. beta-arrestin 2 regulates Abeta generation and gamma-secretase activity in Alzheimer's disease. Nat Med 19, 43-49, doi:10.1038/nm.3023 (2013); Urs, N. M. et al. Targeting beta-arrestin2 in the treatment of L-DOPA-induced dyskinesia in Parkinson's disease. Proc Natl Acad Sci USA 112, E2517-2526, doi:10.1073/pnas.1502740112 (2015); Zhu, L. et al. beta-arrestin-2 is an essential regulator of pancreatic beta-cell function under physiological and pathophysiological conditions. Nat Commun 8, 14295, doi:10.1038/ncomms14295 (2017); and Fereshteh, M. et al. beta-Arrestin2 mediates the initiation and progression of myeloid leukemia. Proc Natl Acad Sci USA 109, 12532-12537, doi:10.1073/pnas.1209815109 (2012)). In particular, βarrs are implicated in the initiation and progression of cancer phenotypes, including prostate and ovarian cancer, non-small cell lung cancer, chronic myelogenous leukemia, and glioblastoma (Fereshteh, M. et al. beta-Arrestin2 mediates the initiation and progression of myeloid leukemia. Proc Natl Acad Sci USA 109, 12532-12537, doi:10.1073/pnas.1209815109 (2012); Rosano, L. et al. Beta-arrestin links endothelin A receptor to beta-catenin signaling to induce ovarian cancer cell invasion and metastasis. Proc Natl Acad Sci USA 106, 2806-2811, doi:10.1073/pnas.0807158106 (2009); Song, Q., et al., The role and mechanism of betaarrestins in cancer invasion and metastasis (Review). Int J Mol Med 41, 631-639, doi:10.3892/ijmm.2017.3288 (2018); Rein, L. A. et al. beta-Arrestin2 mediates progression of murine primary myelofibrosis. JCI Insight 2, doi:10.1172/jci.insight.98094 (2017); Sobolesky, P. M. & Moussa, O. The role of beta-arrestins in cancer. Prog Mol Biol Transl Sci 118, 395-411, doi:10.1016/B978-0-12-394440-5.00015-2 (2013); and Cong, L. et al. Loss of beta-arrestin-2 and Activation of CXCR2 Correlate with Lymph Node Metastasis in Non-small Cell Lung Cancer. J Cancer 8, 2785-2792, doi:10.7150/jca.19631 (2017)). Thus, selective inhibition of βarr function could provide a novel therapeutic framework to fine-tune receptor-βarr signaling. To date, there are no established small-molecule compounds that directly bind βarr1 or βarr2, in contrast with the numerous clinical drugs targeting GPCRs.

Herein, using a multi-tiered approach, novel small-molecules that directly bind to β-arrestins and modulate their activity are characterized. Biophysical screening of drug-like small molecules in vitro, followed by hit characterization through integrated biochemical, pharmacologic, functional, and structural approaches are disclosed. Four chemically-distinct lead compounds (Cmpd-30, -5, -46, and -64) bind both βarr1 and βarr2 with high affinity, and induce conformational changes in the protein that allosterically inhibit βarr-GPCR interaction. Surprisingly, Cmpd-30 activates downstream MAP kinase signaling in a receptor-independent manner. Cmpd-30 can stabilize β-arrestin as a homo-oligomer (dimer/trimer) via an allosteric mechanism. The results herein provide for the development of small molecules for use as both research probes to study the function of β-arrestins and as potential therapeutic agents in disease states where arrestin function is dysregulated.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides, in part, novel compounds that are small molecule modulators of β-arrestins (βarrs), and methods of using said compounds in the diagnosis and treatment of disease states involving βarrs.

One aspect of the present disclosure provides a compound comprising, consisting of, or consisting essentially of the general formula (I) (termed Cmpd 5):

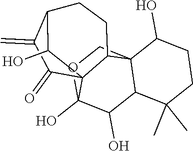

or a pharmaceutically acceptable salt, solvate, hydrate, prodrug, or derivative thereof.

Another aspect of the present disclosure provides a compound comprising, consisting of, or consisting essentially of the general formula (I) (termed Cmpd 30):

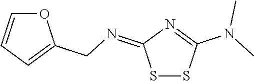

or a pharmaceutically acceptable salt, solvate, hydrate, prodrug, or derivative thereof.

Another aspect of the present disclosure provides a compound comprising, consisting of, or consisting essentially of the general formula (I) (termed Cmpd 46):

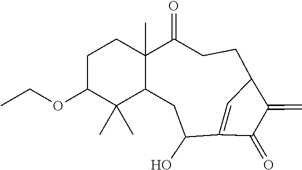

or a pharmaceutically acceptable salt, solvate, hydrate, prodrug, or derivative thereof.

Another aspect of the present disclosure provides a compound comprising, consisting of, or consisting essentially of the general formula (II) (termed Cmpd 64):

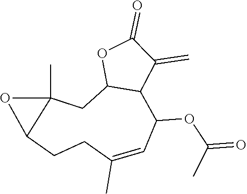

or a pharmaceutically acceptable salt, solvate, hydrate, prodrug, or derivative thereof.

Another aspect of the present disclosure provides a compound comprising, consisting of, or consisting essentially of the general formula (II) (termed Cmpd B29):

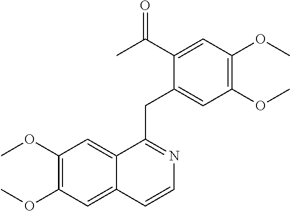

or a pharmaceutically acceptable salt, solvate, hydrate, prodrug, or derivative thereof.

Another aspect of the present disclosure provides a compound comprising, consisting of, or consisting essentially of the general formula (III) (termed Cmpd 31):

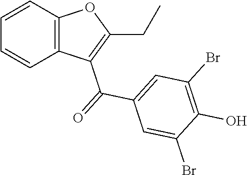

or a pharmaceutically acceptable salt, solvate, hydrate, prodrug, or derivative thereof.

Another aspect of the present disclosure provides a compound comprising, consisting of, or consisting essentially of the general formula (IV) (termed Cmpd 32):

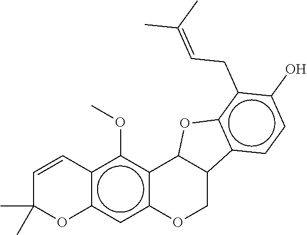

or a pharmaceutically acceptable salt, solvate, hydrate, prodrug, or derivative thereof.

Another aspect of the present disclosure provides a pharmaceutical composition comprising, consisting of, or consisting essentially of a compound as described herein and a pharmaceutically acceptable carrier and/or excipient.

Yet another aspect of the present disclosure provides a method of modulating β-arrestin (βarr) activity in a cell and/or subject comprising, consisting of, or consisting essentially of administering to the cell and/or subject an effective amount of a compound as provided herein such that the βarrestin (βarr) activity is modulated in the cell and/or subject.

Another aspect of the present disclosure provides a method of inhibiting βarr activity in a cell and/or subject, the method comprising, consisting of, or consisting essentially of administering to the cell and/or subject an effective amount of a compound selected from the group consisting of Cmpd 30, Cmpd 29 (also referred to as Cmpd B29) and combinations thereof such that the βarr activity is inhibited in the cell and/or subject.

Another aspect of the present disclosure provides a method of activating βarr activity in a cell and/or subject, the method comprising, consisting of, or consisting essentially of administering to the cell and/or subject an effective amount of a compound selected from the group consisting of Cmpd 31, Cmpd 32 and combinations thereof such that the βarr activity is activated in the cell and/or subject.

Another aspect of the present disclosure provides a method of treating a βarr-associated disease in a subject, the method comprising, consisting of, or consisting essentially of administering to the subject a therapeutically effective amount of a compound as provided herein such that the βarr-associated disease is treated in the subject.

In some embodiments, the βarr-associated disease is selected from the group consisting of cancer, asthma, metabolic diseases, chronic pain, cardiovascular diseases, neurological diseases, and combinations thereof.

Another aspect of the present disclosure provides a method of inhibiting chemotaxis of T cells in a subject, the method comprising, consisting of, or consisting essentially of administering to the subject a therapeutically effective amount of a compound as provided herein such that the chemotaxis of T cells in the subject is inhibited. In some embodiments, the compound comprises Cmpd 30.

Another aspect of the present disclosure provides all that is described and illustrated herein.

### BRIEF DESCRIPTION OF THE DRAWINGS

The foregoing aspects and other features of the disclosure are explained in the following description, taken in connection with the accompanying drawings, herein:

FIG. 1 is an illustration and graphic data representation showing fluorescence-based thermal shift assay (FTSA) screening of βarrestin-compound interaction in vitro. FTSA allows the measurement of physical interactions between a ligand and a protein of interest by virtue of the ability of the ligand to either increase or decrease the stability and temperature sensitivity of the folded protein conformational state. The Tₘ is determined by plotting the increase in temperature at which each melt curve has 50% fraction of the protein in the unfolded state. Tm can also be determined from first derivative fluorescence emission plots as a function of temperature [−dF/dT]). The difference between Tm of the protein-ligand complex and the Tm of the apo protein represents the thermal shift (ΔTₘ), which is a measure of ligand binding to a protein of interest. A stabilizer compound would have a positive ΔTₘ (as in compound ‘B’, leading to a rightward shift in the unfolding transition relative to the protein alone, middle curve) or negative (as in compound ‘A’, leading to a leftward shift in the unfolding transition relative to the protein alone, middle curve).

FIGS. 2A and 2B are graphic representations showing assay validation using three known βarrestin binders: V2Rpp (a phosphorylated C-terminal version of a GPCR), and two endogenous ligands, IP₆ and Heparin. Using the FTSA method, βarr1 is observed to have a Tₘ of approximately 54.5° C. (FIG. 2A) while βarr2 49° C. (FIG. 2B), both in the absence of any ligand. Upon addition of V2Rpp, IP6, and Heparin the unfolding transition of both βarr is shifted to significantly higher or lower temperatures (≥2° C. or ≤−2° C.; shown by dashed lines), implying binding of the known binder to the natively folded βarr1/2. Binders are shown binding to βarr1 in FIG. 2A and to βarr2 in FIG. 2B, both as assessed by thermal shift assay.

FIG. 3 is a flow chart illustrating identification of βarrestin binding small molecule modulators using FTSA in vitro. Approximately 3,500 structurally diverse, drug-like compounds (DDLC, see Materials and Methods) were screened against purified βarr1 or βarr2 at a compound concentration of 50 μM. The primary screen identified 80 hits that altered the thermal conformational stability of βarr1 or βarr2 by 2° C. compared to controls. Based on secondary confirmation binding, activity and toxicity assays, the 80 initial hits were reduced to 56 hits to undergo further characterization.

FIGS. 4A and 4B are graphic representations showing FTSA-based binding of hits to βarrestin-1 or -2. Plots of the change in melting thermal shift (ΔTm) of βarrs (βarr1 open bar graphs, βarr2 closed bar graphs) in presence of hit compounds (total 56 small-molecules). V2Rpp is a control βarrs-binding phosphorylated peptide which corresponds to the C-terminus of the GPCR, vasopressin-2 receptor (V2R). Compounds scoring ΔTm values ≥2 or ≤−2° C. were considered potential binders (dashed lines above and below 0). FIG. 4A, Compounds C1-C40; FIG. 4B, Compounds C41-C79.

FIGS. 5A and 5B are graphic representations showing the effect of putative βarrestin binding compounds on βarr recruitment to agonist-activated GPCR. DiscoveRx-U2OS cells exogenously expressing βarr2 and β2V2R were treated with each putative βarrestin binding compound at 50 μM for 30 min and then stimulated with agonist isoproterenol (10 nM) to induce recruitment of βarr as described in methods. Data are presented as means±SEM (n=5). Dashed lines indicate control agonist alone mediated βarrestin recruitment to the GPCR, above which in presence of compounds indicates compound that enhance βarrs activity while below which inhibit βarr activity. Representative activators (4 compound in shaded dashed line boxes) and inhibitors (12 compound in dashed line boxes). FIG. 5A, Compounds C1-C40; FIG. 5B, Compounds C41-C79.

FIGS. 6A and 6B are graphic representations showing the effect on βarr-promoted high agonist binding-affinity state of receptor in vitro. All 56 compounds were evaluated for their influence on βarr1 or 2-promoted high-affinity receptor state in radio-labeled agonist (³H-Fenoterol, ‘³H-Fen’) binding studies in vitro, using phosphorylated GPCR, β2V2R in membranes. Binding of an agonist at the orthosteric pocket of GPCRs has been previously shown to promote enhanced binding affinity of the βarrs as well as the bound agonist for the receptor. Here, the exogenously added βarrs (second bar-graph from left; βarr1 in panel A, βarr2 in panel B) enhanced the high-affinity agonist (³H-Fen) binding state of the pβ2V2R (open bar graphs in both FIGS. 6A and 6B). Inhibitors decrease while activators increase this βarr-promoted high-affinity ³H-Fen binding signals (bar-graphs in black). The first bar graph (from left) in each panel indicates is DMSO alone without βarr1 or βarr2. Dashed lines (in both FIGS. 6A and 6B) indicate control lines, above which indicates compound that activate and below which compound that inhibit βarrs.

FIG. 7A shows chemical structures of 4 βarrestin inhibitors. All of these 4 compounds, ‘activators’ enhance receptor-agonist promoted β-arrestin activities by more than 50% of that induced by isoproterenol (see FIGS. 4A and 4B on the effects of these 4 compound on β-arrestin recruitment to agonist receptor and β-arrestin 1/2 promoted high affinity agonist state of active receptor).

FIG. 7B shows chemical structures of 12 βarrestin inhibitors. Of the 12 confirmed βarr-inhibitors hits, four compounds (Cmpd-5, -30, -46, and -64) are of particular interest due to their binding capacity to both βarr-isoforms and their inhibition of βarr activity.

FIGS. 8A and 8B are graphic representations showing the effect of 12 putative βarr inhibitors on βarr recruitment to agonist activated GPCR (FIG. 8A: C1, C5, C18, C26, C29, and C30; FIG. 8B: C38, C41, C42, C46, C64, and C74). The data shows validations of 12 putative βarr-inhibitors for their effect on recruitment of βarr2 to activated β2V2R. DiscoveRx-U2OS cells exogenously expressing βarr2 and β2V2R were treated with each putative βarrestin binding compound at 50 μM for 30 min and then stimulated with increasing concentrations of isoproterenol (agonist for the GPCR β2AR) to induce recruitment of βarr as described in methods. Arrow indicates relative difference between the inhibitor treated curves versus control (agonist alone dose-response curve).

FIGS. 9A and 9B are graphic representations showing the effect of 12 putative βarr binding inhibitors on βarr mediated receptor internalization (FIG. 9A: C1, C5, C18, C26, C29, and C30; FIG. 9B: C38, C41, C42, C46, C64, and C74). Figure shows validations of 12 putative βarr-inhibitors for their effect on for their effect on βarr mediated receptor internalization. DiscoveRx-U2OS cells exogenously expressing βarr2 and β2V2R were treated with each putative βarrestin binding compound at 25 μM for 30 min and then stimulated with increasing concentrations of isoproterenol (agonist for the GPCR β2AR) to induce receptor-βarr complex internalization as described in methods. Arrow indicates relative difference between the inhibitor treated curves versus control (agonist alone dose-response curve).

FIGS. 10A-10E are graphic representations showing compounds (Cmpds 5, B29 (also referred to herein as C29) [29 to be added from prov] C30, C46, and C64, respectively) that inhibit agonist-promoted βarrestin recruitment to activated GPCR in a dose-dependent fashion. For each compound, effects on agonist dose-response-curves in βarr recruitment assay are shown. DiscoveRx U2OS cells were pretreated with indicated concentrations of compounds for 30 min and then stimulated with a range of isoproterenol concentrations. Treatment of cells with a series of concentrations of C5, C29, C30, C46 and C64 significantly diminished the maximal agonist induced βarr recruitment responses.

FIGS. 11A-11D are graphic representations showing compounds (Cmpds 5, 30, 46, and 64, respectively) that inhibit βarrestin-mediated GPCR internalization/endocytosis. For each compound, effects of compounds on agonist dose-response-curves in βarrestin-receptor internalization are shown. DiscoveRx U2OS cells were pretreated with indicated concentrations of compounds for 30 min and then stimulated with a range of isoproterenol concentrations as described in methods. Treatment of cells with a series of concentrations of C5, C30, C46 and C64 significantly diminished the maximal agonist induced receptor-βarr internalization responses.

FIG. 12 is a graphic representation showing that βarrestin inhibitors reduce receptor association with early endosomes. HEK 293T cells transiently expressing V2R-RlucII and 2×FYVE-mVenus (that associates with early endosomes) were incubated with vehicle or with indicated βarr inhibitor compounds for thirty minutes, and subsequently stimulated with AVP and read BRET as described in methods. An increase in the net BRET ratio in this assay indicates RlucII-tagged GPCR association with early endosomes.

FIGS. 13A-13D are graphic representations showing that βarr inhibitors slow the rate of agonist-stimulated receptor desensitization. Agonist-induced cAMP (FIGS. 13A and 13B) and calcium (FIGS. 13C and 13D) signals in β2AR and AT1aR systems in presence or absence of inhibitors to measure their effects on respective receptor desensitization. Kinetics of agonist (ISO, 1004, FIGS. 13A and 13B) induced cAMP signal using ICUE2 FRET sensor expressing HEK 293 cells and calcium signal (agonist AngII, 10 pM, FIGS. 13C and 13D) in AT1aR expressing HEK293 cells. FIGS. 13B and 13D show quantification (area under the curve, AUC) of extent of agonist induced second messenger generation in absence or presence of indicated inhibitor.

FIGS. 14A-14C are photographs and graphic representations showing that Cmpd30-activates ERK in a receptor independent manner while the other three attenuate ERK activation through a GPCR, β2-adrenergic receptor. (FIG. 14A) Effect of Cmpd-5, -30, -46 and -64 on carvedilol-induced β₂AR-mediated ERK phosphorylation in HEK293 cells stably expressing FLAG-tagged β₂ARs. Bar graphs (FIG. 14B) showing quantification of ERK activation in presence of vehicle DMSO, 1 μM agonist isoproterenol (ISO), 10 μM of a βarr biased ligand Carvedilol (Cary), 30 μM the compounds (Cmpd-5, -30, -46, or -64) alone or together with Carvedilol (Cary). HEK293 cells stably expressing FLAG-tagged β₂ARs were pretreated with vehicle or compound for 30, then stimulated with indicated concentration of carvedilol for 5 min as detailed in methods section. Data represent the mean±SEM for n independent experiments. DMSO no stimulation; Cary carvedilol; Iso isoproterenol; p-ERK phosphorylated ERK; t-ERK total ERK. (FIG. 14C) and (FIG. 14D) Cmpd30-activates ERK in a β-arrestin dependent manner. The effect of β-arrestin knockdown on Cmpd30-stimulated ERK phosphorylation. HEK293 cells (β₂AR stable cells) with transfection of control siRNA or β-arrestin1/2 siRNA were pretreated with vehicle, EGF (control) or Cmpd-30 for series of time points. Cmpd-30-induced ERK phosphorylation was diminished by β-arrestins siRNA (as shown in Western blot images in FIG. 14C or quantification in FIG. 14D) suggesting the requirement of β-arrestins for this signaling.

FIGS. 15A and 15B are graphic representations showing that Cmpd-30 impairs chemotaxis of wild-type mouse T-cells in response to the chemokine CCL19. βarrestins scaffold multiple proteins that control cell polarity and influence cellular migration downstream of GPCRs, including the chemokine receptors. (FIG. 15A: Helper T cell) and (FIG. 15B: Cytotoxic T cell) To investigate if Cmpd-30 influenced a complex cellular function known to require βarrestins, its effect on T cell chemotaxis were tested. Consistent with its ability to inhibit βarr activity, Cmpd-30, significantly impaired chemotaxis of wild-type mouse T cells.

FIGS. 16A and 16B are a graphic representation and micrographs, respectively showing that Cmpd-30 promotes homo-oligomerization (dimers/trimers) of βarr2, including biophysical analysis and molecular architecture of βarr-Cmpd30 complex. βarr2-forms homo-oligomers in presence of Cmp30 as assessed by dynamic light scattering (DLS). FIG. 16A shows particle size distribution analysis by DLS in nm for control and Cmpd-30 treated βarr2. FIG. 16B shows photographs showing EM analysis and molecular architecture of βarr2-Cmpd-30 complex. Micrograph analysis and molecular architecture of βarrestin2-Cmpd30 complexes show that Cmpd30 promotes homo-oligomerization (dimers/trimers) of βarrestin2.

FIG. 17 shows that ERK2 forms a complex with βarr2 in a Cmpd-30 dependent manner. Affinity pull-down of ERK2 using Anti-FLAG M1 Agarose Beads testing for its binding with βarr2 in presence or absence of series concentrations of C30.

### DETAILED DESCRIPTION

βarrs are intimately associated with numerous aspects of GPCR signaling and regulate many downstream events (Luttrell, L. M. & Lefkowitz, R. J. The role of beta-arrestins in the termination and transduction of G-protein-coupled receptor signals. J Cell Sci 115, 455-465 (2002); Lefkowitz, R. J. & Shenoy, S. K. Transduction of receptor signals by beta-arrestins. Science 308, 512-517, doi:10.1126/science.1109237 (2005); Lefkowitz, R. J. Arrestins come of age: a personal historical perspective. Prog Mol Biol Transl Sci 118, 3-18, doi:10.1016/B978-0-12-394440-5.00001-2 (2013); and Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017). Like their G protein counterparts, βarrs mediate various important physiologic functions, including cell development, growth, survival, migration, immune function, neuronal signaling, and protein translation (Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); Gurevich, V. V., Chen, Q. & Gurevich, E. V. Arrestins: Introducing Signaling Bias Into Multifunctional Proteins. Prog Mol Biol Transl Sci 160, 47-61, doi:10.1016/bs.pmbts.2018.07.007 (2018); Gurevich, E. V. & Gurevich, V. V. Arrestins: ubiquitous regulators of cellular signaling pathways. Genome Biol 7, 236, doi:10.1186/gb-2006-7-9-236 (2006)). Accordingly, their aberrant expression or dysregulation has been linked to numerous disorders, such as cancer and inflammatory, metabolic, cardiovascular, and neurologic diseases. Thus, they are considered prime targets for therapeutic intervention. To date, however, no established small-molecule probes directly target βarrs.

To discover small molecules with attractive pharmacologic properties that can specifically modulate βarr signaling activities, a multi-tiered strategy beginning with a primary in vitro high-throughput screening (HTS) platform was used. This primary screening platform, differential scanning fluorimetry (DSF), which is also referred to as fluorescence-based thermal shift assay (FTSA), is a biophysical technique that allowed detection of direct interactions between compounds and βarrs (Vedadi, M. et al. Chemical screening methods to identify ligands that promote protein stability, protein crystallization, and structure determination. Proc Natl Acad Sci USA 103, 15835-15840, doi:10.1073/pnas.0605224103 (2006); Renaud, J. P. et al. Biophysics in drug discovery: impact, challenges and opportunities. Nat Rev Drug Discov 15, 679-698, doi:10.1038/nrd.2016.123 (2016); Niesen, F. H., et al., The use of differential scanning fluorimetry to detect ligand interactions that promote protein stability. Nat Protoc 2, 2212-2221, doi:10.1038/nprot.2007.321 (2007)). Activity and mechanistic characterization of potential hits by use of a combination of biochemical, pharmacologic, functional, and structural approaches was performed next. Lead modulators among the hits, herein termed Cmpd-5, -30, -46 and -64 emerged as bona fide candidates. All four compounds bound with low nanomolar binding affinity to both isoforms of βarr, having slight selectivity for βarr2. All four βarr inhibitors were shown to prevent the recruitment of βarrs to activated receptor in cell-based functional and in vitro assay formats. Given that recruitment of βarrs to plasma membrane-localized activated receptor typically promotes receptor internalization, the effect of βarr-inhibitors on this βarr-dependent event were also investigated. All four compounds could impair receptor internalization in a dose-dependent manner in different assay formats, demonstrating that inhibition at the transducer level, in this case βarr, can significantly affect critical events in GPCR regulation after agonist activation.

Desensitization of GPCRs after prolonged agonist activation is also regulated by βarrs (Ferguson, S. S. Evolving concepts in G protein-coupled receptor endocytosis: the role in receptor desensitization and signaling. Pharmacol Rev 53, 1-24 (2001); Lefkowitz, R. J. Arrestins come of age: a personal historical perspective. Prog Mol Biol Transl Sci 118, 3-18, doi:10.1016/B978-0-12-394440-5.00001-2 (2013); Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); and Lohse, M. J., et al., beta-Arrestin: a protein that regulates beta-adrenergic receptor function. Science 248, 1547-1550, doi:10.1126/science.2163110 (1990)). They mediate this process by binding to the receptor and sterically inhibiting further receptor-Gα interaction. In these studies, the effect of all four compounds on agonist isoproterenol-induced cAMP production by the prototypical Gαₛ-coupled GPCR, β₂AR, were examined using real-time kinetic measurements (Violin, J. D. et al. beta2-adrenergic receptor signaling and desensitization elucidated by quantitative modeling of real time cAMP dynamics. J Biol Chem 283, 2949-2961, doi:10.1074/jbc.M707009200 (2008)), as well as calcium signaling via angiotensin II type 1a (AT1a) receptor. As expected, treatment with βarr-inhibitors decreased the rate of agonist-stimulated receptor desensitization, leading to significantly higher second messenger generation levels. Results from pharmacologic studies using radiolabeled agonist also agreed with these findings in terms of the mode of action of all of these inhibitors in relation to receptors. That is, all four, potentially via an allosteric mechanism, induced a unique βarr conformation that was incapable of binding and promoting a high-affinity agonist binding state of phosphorylated receptor.

Many drugs targeting GPCRs lack specificity (Hauser, A. S., et al., Trends in GPCR drug discovery: new agents, targets and indications. Nat Rev Drug Discov 16, 829-842, doi:10.1038/nrd.2017.178 (2017); Smith, J. S., et al., Biased signalling: from simple switches to allosteric microprocessors. Nat Rev Drug Discov 17, 243-260, doi:10.1038/nrd.2017.229 (2018); Whalen, E. J., et al., Therapeutic potential of beta-arrestin- and G protein-biased agonists. Trends Mol Med 17, 126-139, doi:10.1016/j.molmed.2010.11.004 (2011); and Violin, J. D., et al., Biased ligands at G-protein-coupled receptors: promise and progress. Trends Pharmacol Sci 35, 308-316, doi:10.1016/j.tips.2014.04.007 (2014)). Depending on the GPCR context, the drugs can produce either adverse or beneficial effects attributed to the downstream GPCR signaling pathways (i.e., G protein or βarr) resulting from the binding of ligands directly at the orthosteric site of the receptor. As a proof-of-concept, blocking βarr function using modulators reduced desensitization and shift the system to signal predominantly through G protein-dependent signaling, underscoring the potential utility of βarr modulators to selectively activate or block subsets of the signaling repertoire of GPCRs. Thus, depending on the GPCR context, this would be a means of achieving functional selectivity or signaling “bias” (i.e., G protein or βarr) of GPCR-balanced ligand responses via targeting βarrs, which would thus eliminate undesired adverse drug effects.

βarrs have been implicated in the initiation and progression of cancer owing to their role in cell migration downstream of GPCRs. This is largely through their ability to scaffold the multiple proteins in actin assembly necessary to form gradient-sensing leading edge protrusions (actin polarization) and directed cell movement (Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); Zoudilova, M. et al. beta-Arrestins scaffold cofilin with chronophin to direct localized actin filament severing and membrane protrusions downstream of protease-activated receptor-2. J Biol Chem 285, 14318-14329, doi:10.1074/jbc.M109.055806 (2010); and McGovern, K. W. & DeFea, K. A. Molecular mechanisms underlying beta-arrestin-dependent chemotaxis and actin-cytoskeletal reorganization. Handb Exp Pharmacol 219, 341-359, doi:10.1007/978-3-642-41199-1_17 (2014)). In this context, Cmpd-30 also inhibits chemokine-induced T cell migration. Treatment of cells with Cmpd-30 eliminated chemotaxis of wild-type mouse T cells in response to stimulation with the chemokine CCL19, consistent with its ability to inhibit βarr activity. These results are consistent with similar studies wherein CCR4-, CXCR3-, and AT1AR-mediated chemotaxis were eliminated in leukocytes obtained from βarr2 knockout mice or in siRNA-based βarr2 knockdown cells (Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); Jiang, D., et al., beta-Arrestins in the immune system. Prog Mol Biol Transl Sci 118, 359-393, doi:10.1016/B978-0-12-394440-5.00014-0 (2013); Hunton, D. L. et al. Beta-arrestin 2-dependent angiotensin II type 1A receptor-mediated pathway of chemotaxis. Mol Pharmacol 67, 1229-1236, doi:10.1124/mol.104.006270 (2005); Lin, R., et al., beta-Arrestin-2-Dependent Signaling Promotes CCR4-mediated Chemotaxis of Murine T-Helper Type 2 Cells. Am J Respir Cell Mol Biol 58, 745-755, doi:10.1165/rcmb.2017-0240OC (2018); and Smith, J. S. et al., Biased agonists of the chemokine receptor CXCR3 differentially control chemotaxis and inflammation. Sci Signal 11, doi:10.1126/scisignal.aaq1075 (2018)). Thus, the observed reduction in cell migration as a result of pharmacologic inhibition of βarr demonstrates the promising possibility of therapeutic intervention in specific cancer types that involve dysregulation of βarr activity.

βarrs can orchestrate a number of intracellular signaling paradigms that occur independent of G protein participation (Lefkowitz, R. J. A brief history of G-protein coupled receptors (Nobel Lecture). Angew Chem Int Ed Engl 52, 6366-6378, doi:10.1002/anie.201301924 (2013); Lefkowitz, R. J. Arrestins come of age: a personal historical perspective. Prog Mol Biol Transl Sci 118, 3-18, doi:10.1016/B978-0-12-394440-5.00001-2 (2013); Gurevich, V. V., et al., Arrestins as multi-functional signaling adaptors. Handb Exp Pharmacol, 15-37, doi:10.1007/978-3-540-72843-6_2 (2008); Peterson, Y. K. & Luttrell, L. M. The Diverse Roles of Arrestin Scaffolds in G Protein-Coupled Receptor Signaling. Pharmacol Rev 69, 256-297, doi:10.1124/pr.116.013367 (2017); Luttrell, L. M. et al. Manifold roles of beta-arrestins in GPCR signaling elucidated with siRNA and CRISPR/Cas9. Sci Signal 11, doi:10.1126/scisignal.aat7650 (2018); Luttrell, L. M. et al. Activation and targeting of extracellular signal-regulated kinases by beta-arrestin scaffolds. Proc Natl Acad Sci USA 98, 2449-2454, doi:10.1073/pnas.041604898 (2001); Smith, J. S., et al., Biased signalling: from simple switches to allosteric microprocessors. Nat Rev Drug Discov 17, 243-260, doi:10.1038/nrd.2017.229 (2018); Whalen, E. J., et al., Therapeutic potential of beta-arrestin- and G protein-biased agonists. Trends Mol Med 17, 126-139, doi:10.1016/j.molmed.2010.11.004 (2011); Violin, J. D., et al., Biased ligands at G-protein-coupled receptors: promise and progress. Trends Pharmacol Sci 35, 308-316, doi:10.1016/j.tips.2014.04.007 (2014); Wisler, J. W. et al. A unique mechanism of beta-blocker action: carvedilol stimulates beta-arrestin signaling. Proc Natl Acad Sci USA 104, 16657-16662, doi:10.1073/pnas.0707936104 (2007); Wootten, D., et al., Mechanisms of signalling and biased agonism in G protein-coupled receptors. Nat Rev Mol Cell Biol 19, 638-653, doi:10.1038/s41580-018-0049-3 (2018); Kahsai, A. W. et al. Multiple ligand-specific conformations of the beta2-adrenergic receptor. Nat Chem Biol 7, 692-700, doi:10.1038/nchembio.634 (2011); Xiao, K. et al. Global phosphorylation analysis of beta-arrestin-mediated signaling downstream of a seven transmembrane receptor (7TMR). Proc Natl Acad Sci USA 107, 15299-15304, doi:10.1073/pnas.1008461107 (2010); and Noma, T. et al. Beta-arrestin-mediated beta1-adrenergic receptor transactivation of the EGFR confers cardioprotection. J Clin Invest 117, 2445-2458, doi:10.1172/JCI31901 (2007)). βarrs are known to mediate ERK1/2 activation by serving as receptor agonist-regulated scaffolds for several signaling components, including the cRaf1-MEK1/2-ERK1/2 MAP kinase cascade (Luttrell, L. M. et al. Manifold roles of beta-arrestins in GPCR signaling elucidated with siRNA and CRISPR/Cas9. Sci Signal 11, doi:10.1126/scisignal.aat7650 (2018); and Luttrell, L. M. et al. Activation and targeting of extracellular signal-regulated kinases by beta-arrestin scaffolds. Proc Natl Acad Sci USA 98, 2449-2454, doi:10.1073/pnas.041604898 (2001))^{17,18}. The consequences of pharmacologic inhibition of βarr recruitment to GPCRs on βarr-dependent ERK activation downstream of GPCRs were investigated. Surprisingly, Cmpd-30 dose-dependently activated ERK1/2 (EC₅₀=500 nM) in a βarr-dependent but receptor-independent manner. This finding was paradoxical because Cmpd-30 inhibited most receptor-related functions of βarr, including βarr recruitment, receptor desensitization, internalization, and GPCR-mediated βarr-dependent migration of cells. Thus, this signaling profile is not in agreement with the traditional GPCR signaling paradigm, in which downstream signaling profiles (such as βarr-dependent ERK1/2 phosphorylation, for example) should typically be abrogated upon antagonist binding at a receptor. Notably, the findings herein show that Cmpd-30 facilitates βarr2 and ERK2 interaction and suggest that Cmpd-30 may be able to promote unique conformational changes on βarrs via allosteric modulation that allow them to have high-affinity interaction with ERK.

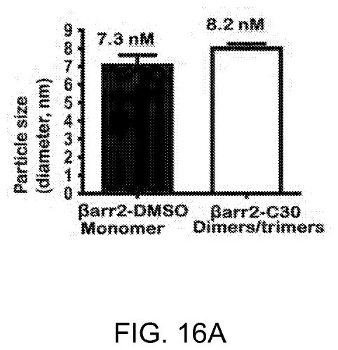

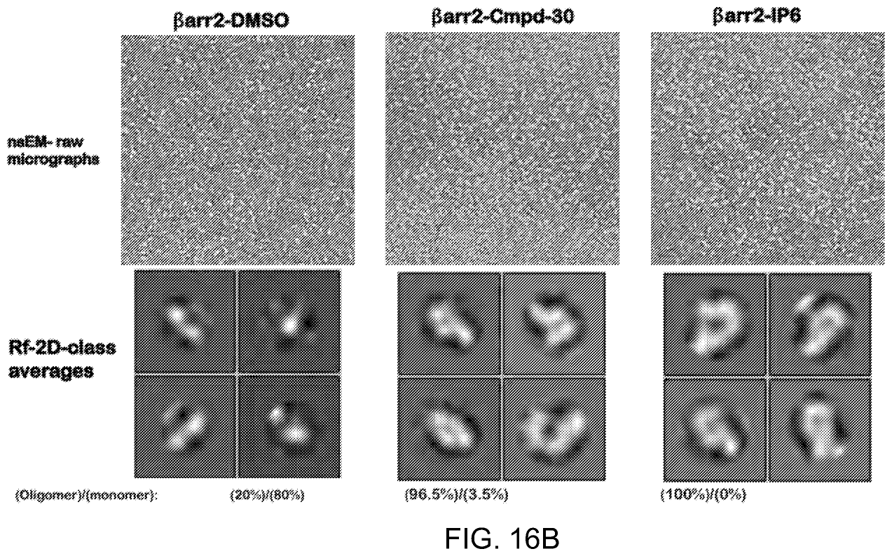

The results of the biophysical and structural studies disclosed herein shed light on the mechanism by which Cmpd-30 modulates βarr function to promote ERK1/2 activity. First, using single-particle analysis of negatively stained EM images of βarr2 samples, it was shown that Cmpd-30 promotes homo-oligomerization of βarr, primarily in dimer and trimer states. Second, DLS analysis was used to further validate these finding (FIG. 16A). The single-particle EM averages suggested that oligomerization was mediated by the interaction between the βarr domain lobes (N and C), for which the oligomerization domain of one sister protomer interacted with another, leading to stabilized interactions that allowed the formation of homo-oligomeric units. The precise molecular mechanism by which Cmpd-30 promotes homo-oligomerization of βarrs, however, must be the subject of future studies (e.g., using cryoEM). Thus, formation of homo-oligomeric structures of βarrs in the presence of Cmpd-30 likely explains the ability of βarrs to mediate ERK activity independent of receptor, potentially by allowing these homo-oligomers to act as distinct scaffolds that connect with unique signaling cargos, hence defining a novel, previously unappreciated scaffolding role of βarrs. The lack of βarr recruitment to receptor and high-affinity agonist state in the presence of Cmpd-30 can thus be explained by the current mechanism whereby the homo-oligomers of βarr are unable to bind to agonist-activated receptor states owing to steric effects (hindrance), even though each sister protomer in the oligomer may still likely be in the active state of βarr as previously reported. More broadly, the results herein imply that, in a complex and crowded cellular milieu, such self-association of βarrs is likely a unique property whereby under specific cellular cues, βarrs can adopt distinct conformational states (monomeric or homo-oligomeric) that allow each to provide a high-avidity interaction surface to select signaling units and thus mediate diverse signaling nodes.

βarrs have also been shown previously to form homo-oligomeric complexes in the presence of a highly negatively charged cellular metabolite, inositol hexakisphosphate (IP6)(Boularan, C. et al. beta-arrestin 2 oligomerization controls the Mdm2-dependent inhibition of p53. Proc Natl Acad Sci USA 104, 18061-18066, doi:10.1073/pnas.0705550104 (2007); and Chen, Q. et al. Structural basis of arrestin-3 activation and signaling. Nat Commun 8, 1427, doi:10.1038/s41467-017-01218-8 (2017)). Interestingly, such IP6-bound homotrimer forms of βarr2 have also been reported to enhance βarr scaffolding of the ASK1-MKK4/7-JNK3 cascade and facilitate receptor-independent JNK3 activation (Chen, Q. et al. Structural basis of arrestin-3 activation and signaling. Nat Commun 8, 1427, doi:10.1038/s41467-017-01218-8 (2017); and Song, X., Coffa, S., Fu, H. & Gurevich, V. V. How does arrestin assemble MAPKs into a signaling complex? J Biol Chem 284, 685-695, doi:10.1074/jbc.M806124200 (2009)), similar to the mode of interaction and activation of ERK1/2 we observed in this study. These and the results herein show that βarrs indeed form organized lower-order oligomers and use these as scaffolds for signaling components. This differs from the prevailing notion that the scaffolding role of βarr requires only a monomeric active form of βarr and that the receptor has to be present in the complex. In fact, recent studies have shown that βarrs can remain active alone after dissociation from the receptors that activate them and can mediate MAP kinase signaling in the absence of these receptors (Eichel, K., et al., beta-Arrestin drives MAP kinase signalling from clathrin-coated structures after GPCR dissociation. Nat Cell Biol 18, 303-310, doi:10.1038/ncb3307 (2016); Kahsai, A. W., et al., GPCR signaling: conformational activation of arrestins. Cell Res 28, 783-784, doi:10.1038/s41422-018-0067-x (2018); Eichel, K. et al. Catalytic activation of beta-arrestin by GPCRs. Nature 557, 381-386, doi:10.1038/s41586-018-0079-1 (2018); and Nuber, S. et al. beta-Arrestin biosensors reveal a rapid, receptor-dependent activation/deactivation cycle. Nature 531, 661-664, doi:10.1038/nature17198 (2016)). Indeed, the ability of βarrs to adopt distinct conformations indicates the complexity of βarr-dependent signaling (Shukla, A. K. et al. Structure of active beta-arrestin-1 bound to a G-protein-coupled receptor phosphopeptide. Nature 497, 137-141, doi:10.1038/nature12120 (2013); Eichel, K. et al. Catalytic activation of beta-arrestin by GPCRs. Nature 557, 381-386, doi:10.1038/s41586-018-0079-1 (2018); Nuber, S. et al. beta-Arrestin biosensors reveal a rapid, receptor-dependent activation/deactivation cycle. Nature 531, 661-664, doi:10.1038/nature17198 (2016); Lee, M. H. et al. The conformational signature of beta-arrestin2 predicts its trafficking and signalling functions. Nature 531, 665-668, doi:10.1038/nature17154 (2016); Gurevich, V. V. & Gurevich, E. V. Extensive shape shifting underlies functional versatility of arrestins. Curr Opin Cell Biol 27, 1-9, doi:10.1016/j.ceb.2013.10.007 (2014); Scheerer, P. & Sommer, M. E. Structural mechanism of arrestin activation. Curr Opin Struct Biol 45, 160-169, doi:10.1016/j.sbi.2017.05.001 (2017); and Shukla, A. K. et al. Visualization of arrestin recruitment by a G-protein-coupled receptor. Nature 512, 218-222, doi:10.1038/nature13430 (2014)). In fact, some undesired side effects could be the tendency of some of these distinct scaffolding structural units (homo-oligomers and monomers) to form signalosomes. On the other hand, this provides yet another exciting opportunity to optimize therapeutic treatments involving GPCR/βarr signaling.

In summary, using small-molecule βarr-modulators as probes, mechanistic insight into the previously unappreciated scaffolding role of βarrs is provided herein. Specifically, using Cmpd-30, the results herein show that βarr homo-oligomers induce an ERK1/2 MAP kinase signal profile that is distinct from canonical GPCR-mediated downstream responses, a phenomenon that may have translational and clinical implications. In addition to eliciting this scaffolding role and signaling bias, modulators of βarr may have utility in the development of therapeutics for diseases involving βarrs, as demonstrated by the beneficial effects in inhibiting migration of T cells obtained from wild-type mice. The results herein, thus, not only demonstrate the validity of targeting βarr pharmacologically by using small molecules, but also shed critical mechanistic insight into a previously unappreciated biological function of these clinically important regulators.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is a human subject suffering from a condition and/or disease in which the modulation of βarr activity is beneficial to the treatment of said condition and/or disease.

As used herein, the term “β-arrestin” or “βarr” refers to the ubiquitously expressed proteins that are involved in desensitizing G protein-coupled receptors (GPCRs), including all isoforms thereof (e.g., β-arrestin 1 (also referred to as βarr1), β-arrestin 2 (also referred to as βarr2), etc.).

As used herein, the term “β-arrestin-associated disease,” “β-arrestin-associated condition,” “βarr-associated disease,” or “βarr-associated condition” refers to those disease and/or disorders and/or conditions that involve β-arrestin. Examples include, but are not limited to, auto-inflammatory/Inflammatory disorders (e.g., experimental autoimmune encephalomyelitis [EAE], allergic asthma, rheumatoid arthritis, inflammatory bowel disease (IBD), primary biliary cirrhosis, asthma, metabolic diseases, myocardial infarction, pulmonary fibrosis, cystic fibrosis, cutaneous flushing, etc.), Inflammatory responses to pathogens (e.g., endotoxemia, sepsis, meningitis, antiviral responses, etc.), neurological diseases (e.g., Alzheimer's Disease), cancer, metabolic diseases (e.g., diabetes), acute and chronic pain, cancer and the like.

As used herein, the term “cancer” and “cancerous” refers to or describes the physiological condition in mammals that is typically characterized by upregulated/dysregulated cell growth. Examples of cancer include, but are not limited to, carcinoma, lymphoma, blastoma, sarcoma, and leukemia. More particular examples include such cancers as breast cancer, prostate cancer, colon cancer, squamous cell cancer, small cell lung cancer, non-small cell lung cancer, gastrointestinal cancer, pancreatic cancer, glioblastoma, cervical cancer, ovarian cancer, liver cancer, bladder cancer, hepatoma, colorectal cancer, vulval cancer, thyroid cancer, hepatic cancer, and various types of head and neck cancers. In some embodiments, the cancer is characterized by βarr activity.

Unless otherwise defined, all technical terms used herein have the same meaning as commonly understood by one of ordinary skill in the art to which this disclosure belongs.

β-arrestins (βarrs) are versatile adaptor proteins that play central roles in the desensitization and endocytosis of G-protein coupled receptors (GPCRs), as well as signaling independent of or in concert with G-proteins. Accordingly, they influence manifold physiological and pathophysiological processes. To date, the methods used to influence GPCR signaling have been primarily focused at the receptor level via targeting of the primary orthosteric ligand binding site, and there are currently no established small-molecule βarr modulators. Herein, small-molecule modulators that bind β-arrestin (βarr) with nanomolar affinity and disrupt the βarr-GPCR interaction via an unexpected allosteric mechanism are disclosed. These compounds inhibit agonist-promoted GPCR endocytosis and decrease the rate of receptor desensitization. Biophysical, structural, and functional studies reveal the mechanism-of-action, whereby one compound among these, Cmpd-30 promotes functional self-association (homo-oligomerization) of βarrs, which in turn leads to activation of MAP kinase that is distinct from the canonical GPCR-mediated response. Mechanistic insight into previously unappreciated functions of arrestins is provided and underscores the potential of using βarr-targeted small-molecules to attenuate receptor-dependent and -independent activities.

### Primary and Secondary Screens

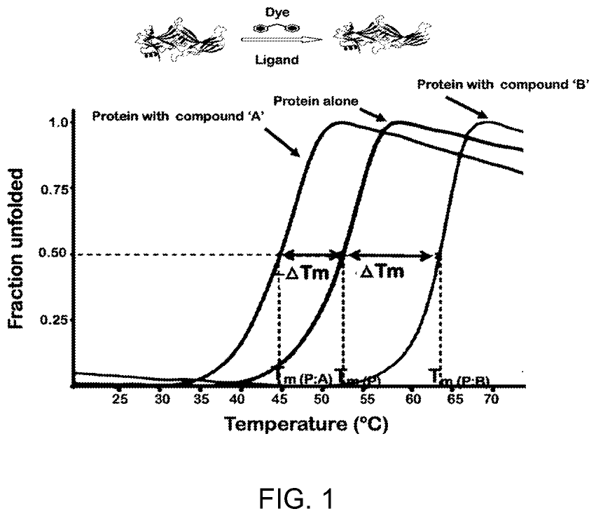

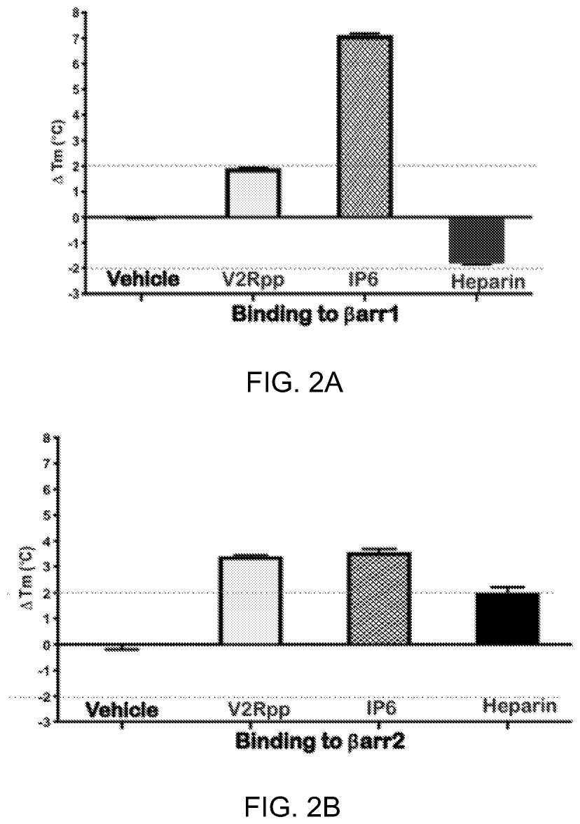

βarrs represent proteins that have topologically distinct sites suitable for perturbation with small molecules. To identify small molecules with attractive pharmacological properties that can modulate βarr receptor dependent and/or independent activity, a multi-tiered strategy was employed, beginning with a primary biophysical platform to screen for hits against purified βarrs. A fluorescence-based thermal shift assay (FTSA) was used to measure changes in the melting temperature (Tm) of purified βarr1 or βarr2 and tested the effects of a small panel that includes ligands, peptides, and antibodies known to bind arrestin. Ligand binding to β-arrestin causes an increase or decrease in the stability and temperature sensitivity of the folded protein conformational state and can be measured by the FTSA approach (see FIG. 1). The panel consists of (i) inositol hexakisphosphate (IP6) and (ii) heparin, both of which highly charged endogenous ligands bind within the N-domain of βarrs, and (iii) V2Rpp, a phosphorylated peptide corresponding to the C-terminus of the vasopressin-2 receptor (V2R) that activates β-arrestin (see FIGS. 2A and 2B). Incubation of purified βarr1 or βarr2 with IP6, heparin, or V2Rpp resulted changes in the stability temperature (Tm) of βarr1 or βarr2, indicative of physical interactions (i.e., binding).

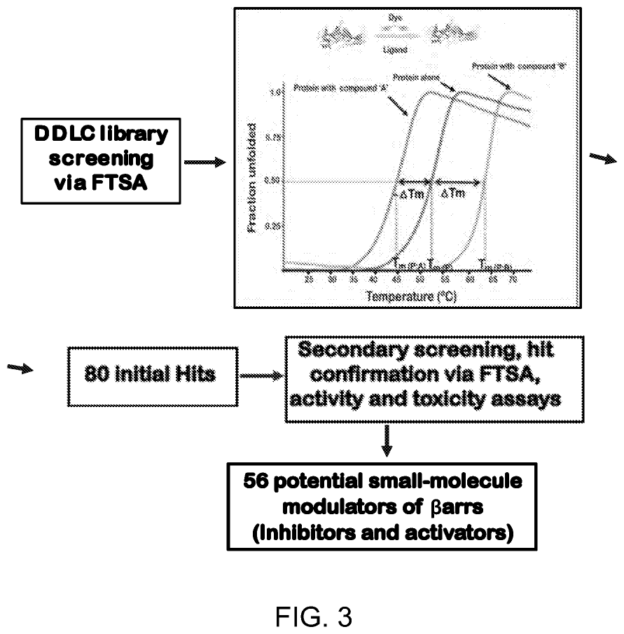

FTSA was used to screen a library of 3,500 structurally diverse, drug-like compounds (DDLC, see Materials and Methods) against purified βarr1 or βarr2 at a compound concentration of 50 μM (see FIG. 3). The primary screen identified 80 hits that altered the thermal conformational stability of βarr1 or βarr2 by 2° C. compared to controls.

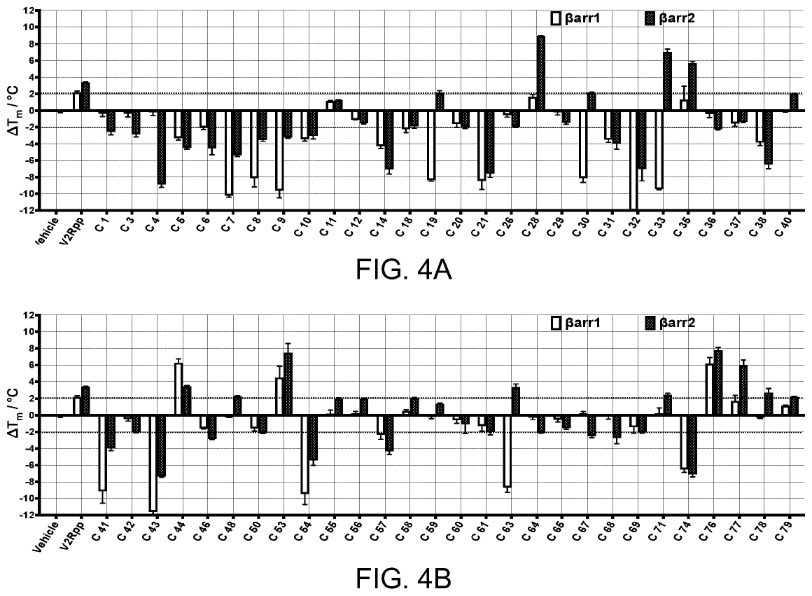

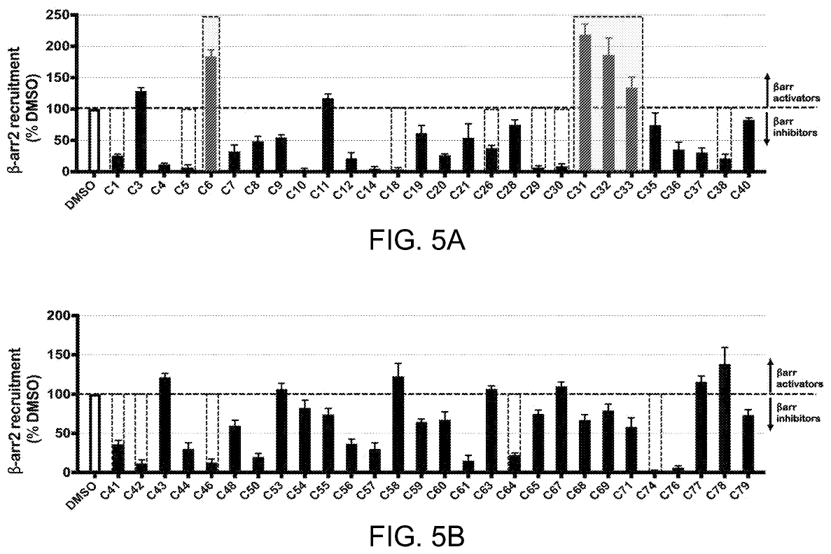

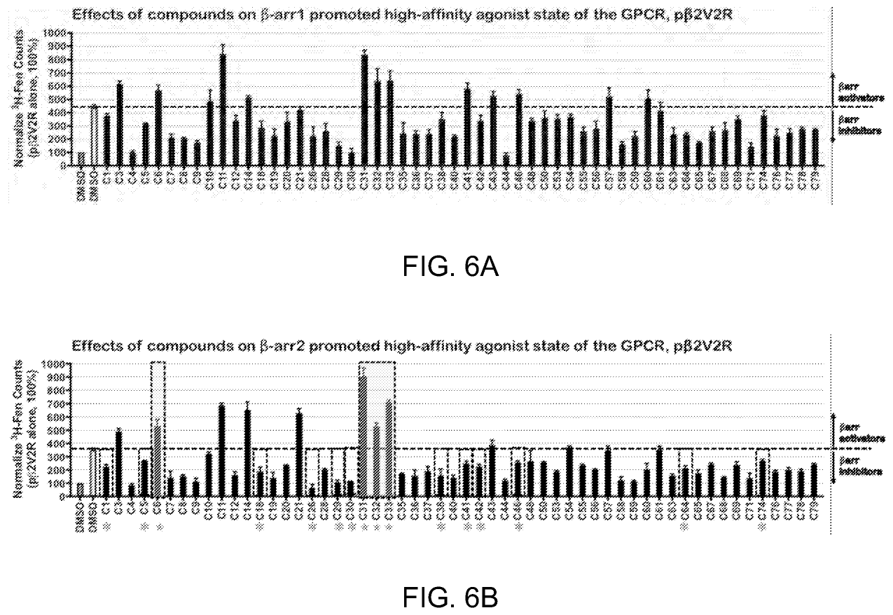

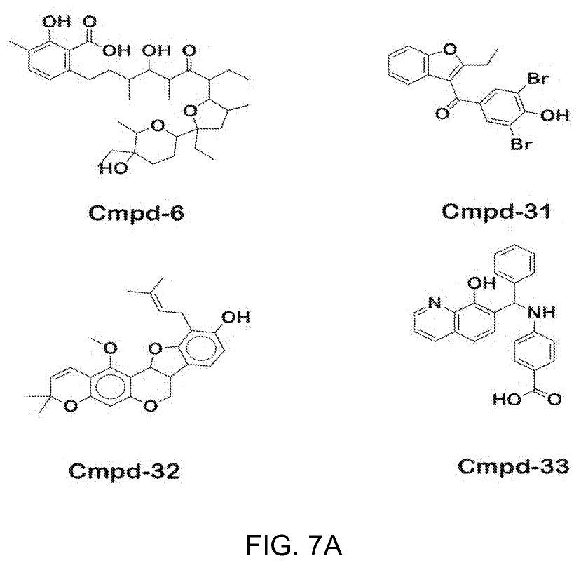

To eliminate false positives caused by assay interference and validate true potential hits, the primary hits were further tested via confirmatory and orthogonal secondary assays. Compounds were evaluated, at a single concentration, based on the following criteria measured: 1) reproducible signal in the affinity-based FTSA (see FIGS. 4A and 4B); 2) ability to modulate agonist-induced βarr2 recruitment to the receptor in a live cell assay (see FIGS. 5A and 5B); and 3) ability to modulate βarr1/2 promoted, high-affinity agonist state of a receptor in a radio-ligand binding assay (see FIGS. 6A and 6B). Based on these criteria, the 80 initial hits were reduced to 56 hits to undergo further characterization (see FIGS. 4A and 4B, FIGS. 5A and 5B, and FIGS. 6A and 6B).

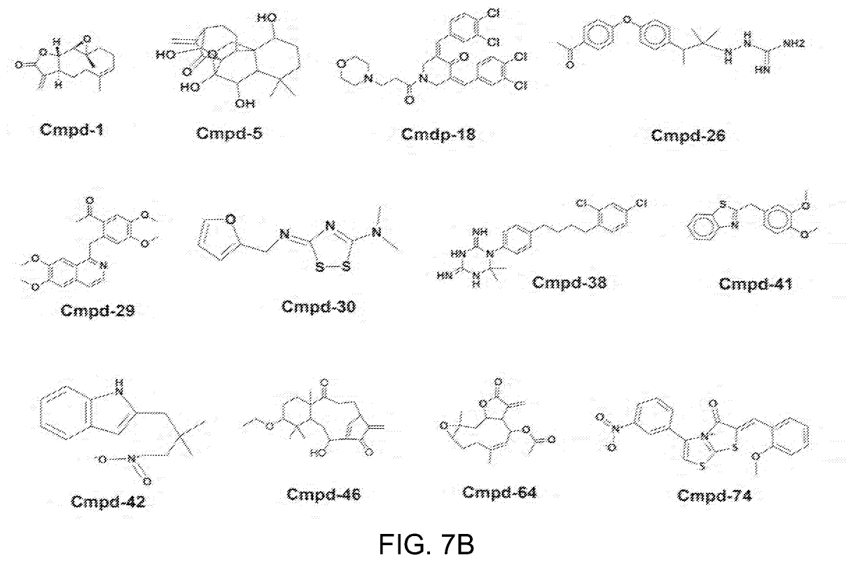

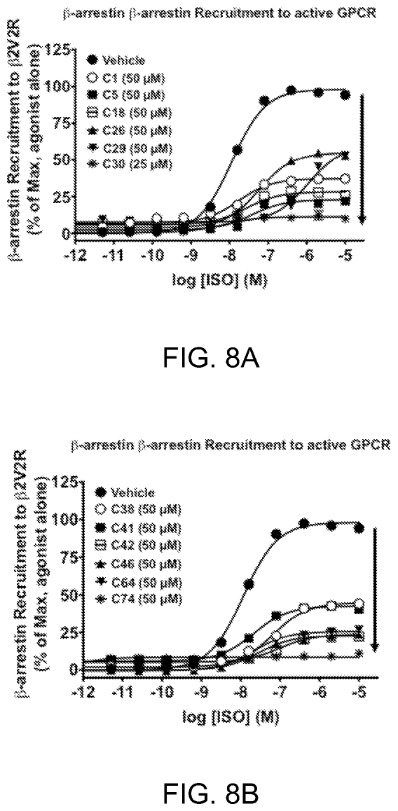

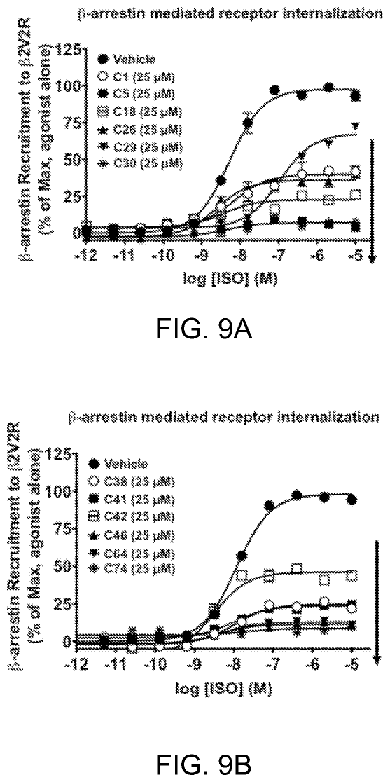

The chemical structures of putative activators (4 compounds) and inhibitors (12 compounds) indicated in boxes (FIGS. 5A and 5B) are shown now FIGS. 7A and 7B, respectively. These 12 putatative βarr-inhibitors were selected for further validation to test their effects on at two critical βarr-mediated receptor activities: βarr-arrestin recruitment to activated receptor and receptor-βarr endocytosis (see FIGS. 8A, 8B and FIGS. 9A and 9B). Of the 12 confirmed βarr-inhibitors hits, four compounds, herein named Cmpd-5, -30, -46, and -64 (NCS: 250682, 622608, 302979, and 22070) were of particular interest due to their binding capacity to both βarr-isoforms and their inhibition of βarr activity (percent inhibition ≥40% as calculated from for the two assays; FIGS. 8A, 8B and FIGS. 9A and 9B); therefore these four inhibitors were selected as lead small molecules for further analyses.

Compounds 5, 30, 46, and 64 Bind with Nanomolar Affinity to β-Arrestins

To assess further the direct interaction of all four compounds with β-arrestin, isothermal titration calorimetry (ITC) was performed. ITC enables measurement of equilibrium binding affinity (K_{d}), binding stoichiometry, and thermodynamic parameters of a protein-ligand interaction. As shown, all four favorably bound to both βarr1 and βarr2 with nanomolar binding affinities.

TABLE 1
Profiles of binding affinities of four compounds to βarr 1 or βarr 2

| | Cmpd-5 | | Cmpd-30 | | Cmpd-46 | | Cmpd-64 | |
|---|---|---|---|---|---|---|---|---|
| | βarr1 | βarr2 | βarr1 | βarr2 | βarr1 | βarr2 | βarr1 | βarr2 |
| K_{d} | 232.9 ± | 260.5 ± | 9.3 ± | 2.0 ± | —^{f} | 411.8 ± | ^{—f} | 103.9 ± |
| (nM)^{a} | 68.26 | 80.39 | 3.5 | 2.5 | | 148.6 | | 38.25 |

^{a}K_{d} is dissociation constant. K_{d} values for Cmpd-30 were determined by ITC and for the other three using a combination of ITC and thermal shift methods.
^{f}Denotes that dissociation constants could not be measured reliably due to poor signal to noise ratios.

Compounds 5, 30, 46, and 64 inhibit β-arrestin recruitment to activated β₂V₂R

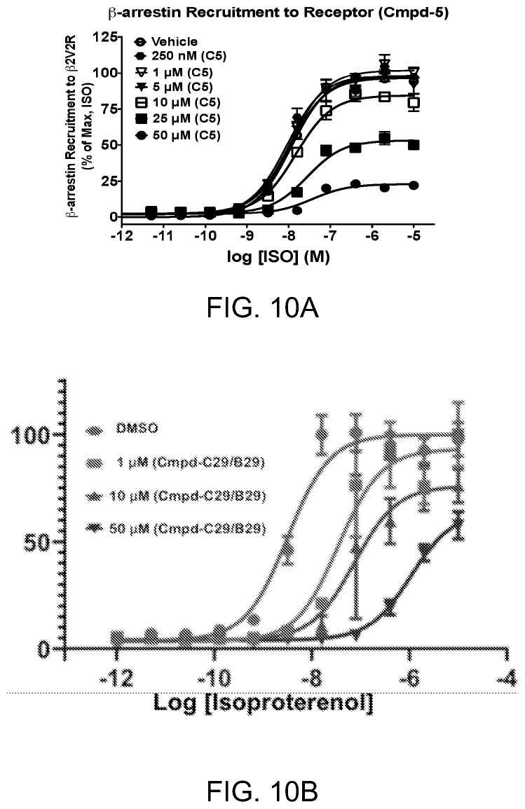

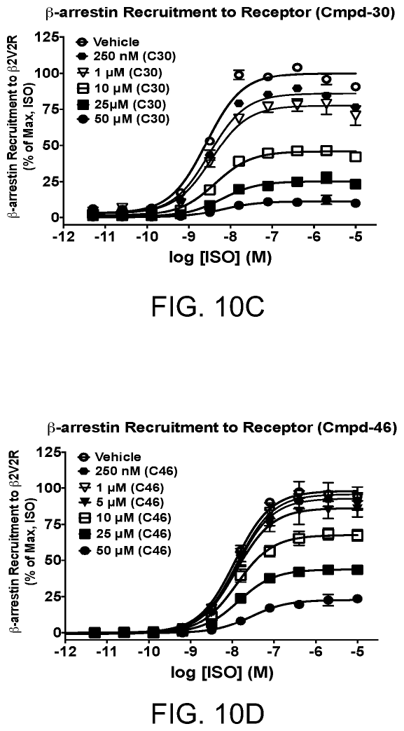

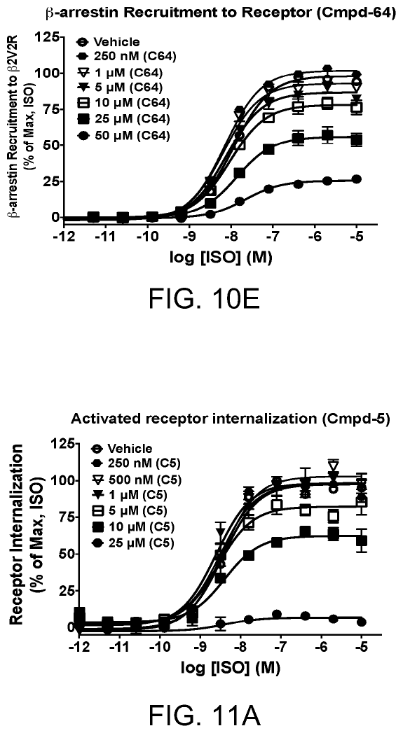

To further characterize the pharmacological properties of compounds 5, 29, 30, 46, and 64, the compound effects on βarr2 recruitment to the activated receptor in cells were measured. A chimeric β₂-adrenergic receptor (β₂AR) with the C-terminal tail substituted from the vasopressin receptor 2 (β₂V₂R) was used (see FIGS. 10A-10E). This construct retains wild-type pharmacological properties and displays stabilized agonist-promoted β-arrestin binding relative to the native β₂AR. Consistent with the single-dose effects observed in FIGS. 5A and 5B and FIGS. 8A and 8B, all four compounds inhibit isoproterenol-induced βarr2 recruitment to receptor in a concentration-dependent manner. A significant decrease in maximal responses by the agonist used was shown for all four inhibitors (Eₘₐₓ from 100% by ISO to 23.0%±1.2%, 11.4%±0.7%, 22.8%±1%, and 25.6%±0.5% for Cmpd-5, -30, -46 and -64, respectively).

Compounds 5, 30, 46, and 64 Inhibit Activated β₂V₂R-β-Arrestin Endocytosis

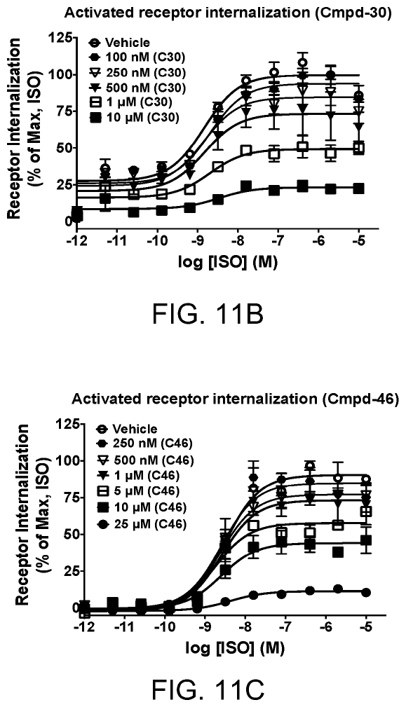

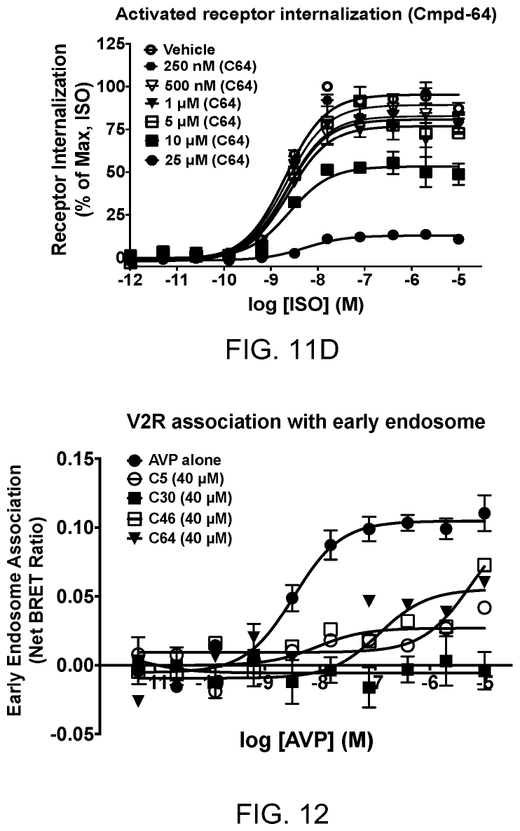

Given that all four compounds inhibit βarr2-recruitment to the activated receptor and that recruitment promotes receptor internalization, or endocytosis, compound effect(s) on this process were examined next. Receptor trafficking is regulated by the cellular location of the arrestin-receptor complex as well as the binding affinity of the arrestin isoform for the receptor. Class A receptors (e.g., β₂AR) have weaker β-arrestin interactions and rapidly recycle back from the endosomes to the cell surface. Class B receptors (e.g., V₂R) have tight interactions with β-arrestin, resulting in a longer residence time in endosomes, before being targeted to lysosomal degradation. The effect of all four on the internalization of a transiently expressed β₂V2R (Class B type receptor) was monitored in U2OS cells stably expressing split (3-galactosidase fragments on βarr2 and endosomes. When the arrestin-bound receptor is internalized into endosomes, β-galactosidase is complemented and provides a luminescent signal upon addition of substrate. Treatment of cells with a series of concentrations of all four compounds significantly diminished the maximal receptor internalization responses (see FIGS. 11A-11D).

Two ‘bystander’ bioluminescence resonance energy transfer (BRET)-based receptor internalization approaches with a different receptor system [V₂R] were also used (see FIG. 12). By measuring the association of labeled receptor with an early endosome marker, an increase in the BRET ratio in V₂R-expressing cells stimulated with arginine vasopressin (AVP) (see FIG. 12) relative to unstimulated cells, indicative of internalization, was observed. Treatment with either compound significantly decreased association between the receptors and the early endosome. As the receptor is internalized, a net decrease in the steady-state levels of the receptor present in the plasma membrane would be expected. Indeed, agonist-stimulation of V₂R results in a decrease in the net BRET ratio between labeled receptor and a plasma membrane marker (see FIGS. 11A-11D). This process was inhibited by all four compounds, consistent with the results from the other cell-based internalization assays (see FIGS. 11A-11D). Collectively, these data demonstrate that compounds 5, 30, 46, and 64 inhibit the internalization of the receptor and their association with early endosomes, both of which are β-arrestin-dependent events.

### βArr Inhibitors Slow the Rate of Agonist-Stimulated Receptor Desensitization

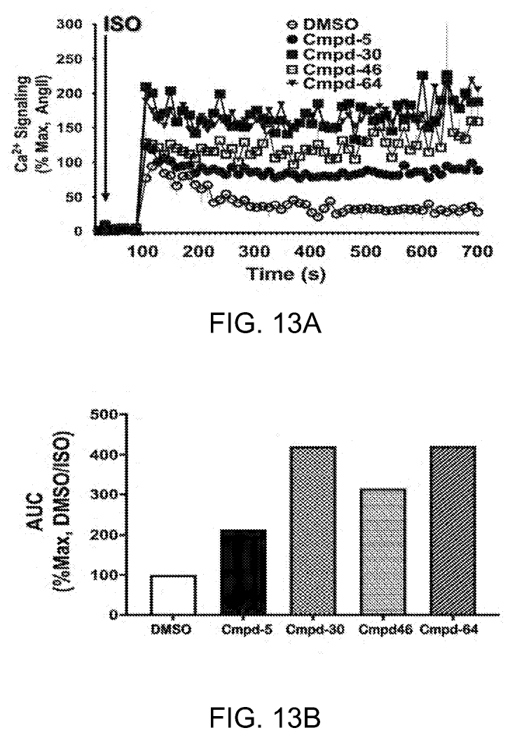

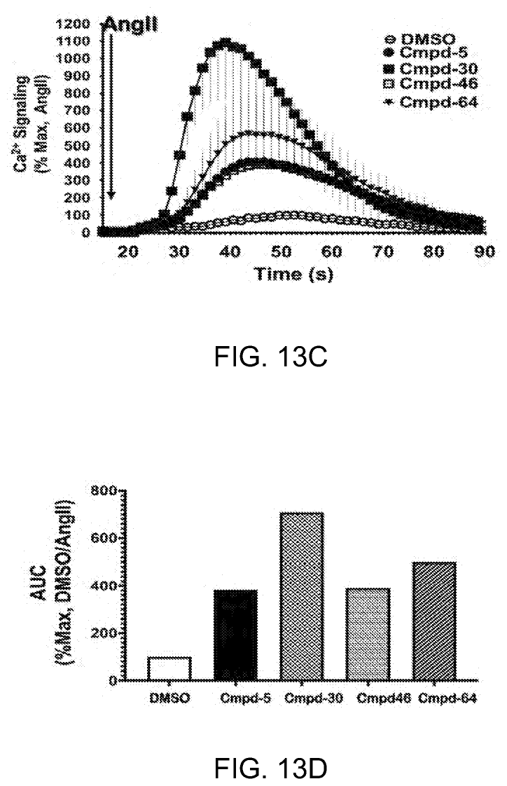

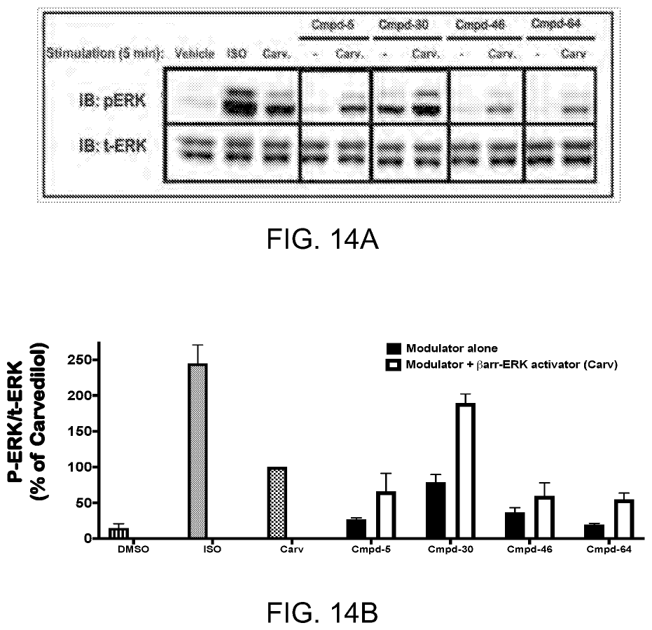

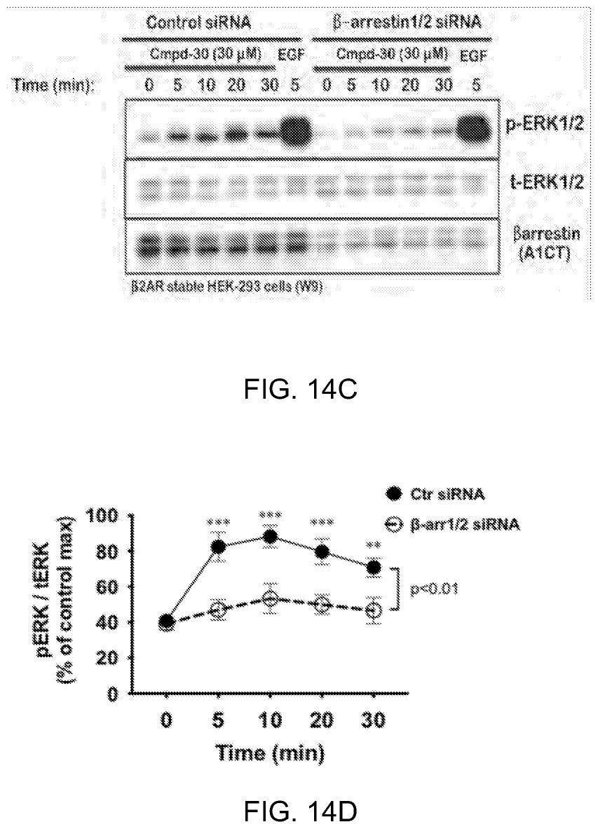

Prolonged activation results in a loss of agonist response that has been attributed to β-arrestin binding and subsequent receptor/Ga uncoupling, followed by desensitization and internalization of the receptor. To investigate possible compound effects on receptor desensitization, fluorescence resonance energy transfer (FRET)-based sensors was used to measure the kinetics of cAMP production following isoproterenol stimulation in control cells and cells pretreated with each inhibitor (see FIGS. 13A and 13B). Cmpd-5, -30, -46, or -64 treatment of the ICUE cells resulted in increased cAMP accumulation as compared to untreated control cells, indicative of sustained receptor/Ga coupling and delayed PAR desensitization (see FIG. 14A). These results demonstrate that all four inhibit the rate of agonist-stimulated β2AR desensitization. Similarly, all four compounds (Cmpd-5, -30, -46, and -64) inhibited the desensitization of AT1aR (see FIGS. 13C and 13D).

Cmpd30-Activates ERK in a Receptor Independent Manner while the Other Three Attenuate ERK Activation Through a GPCR, β2-Adrenergic Receptor.

Next, the effects of β-arrestin inhibitors on signaling downstream of GPCRs were investigated. GPCR-stimulated ERK1/2 activation has been shown to occur in a G protein-independent, but βarr1/2-dependent mechanism. The test was performed by pretreatment of cells with an inhibitor following by agonist stimulation using carvedilol, a β2AR biased agonist that induces moderate ERK1/2 activation in a β-arrestin-dependent manner but antagonizes stimulation of Gas (see FIGS. 14A-14D). The use of a biased ligand allows specific measurement of βarr1/2-dependent signaling and minimizes potential crosstalk between G protein- and arrestin-specific pathways. As expected, three inhibitors (Cmpd-5, -46, and -64) were found to attenuate βarr1/2-dependent ERK activation through the GPCR, β2-adrenergic receptor. Surprisingly, however, one compound among these, Cmpd-30, activates ERK in a receptor independent manner. Treatment with Cmpd-30 alone (absent of agonist) increased the basal phosphorylation of ERK1/2 (see FIG. 14A). This activation is a time-dependent process, with the signal peaking within 15-20 minutes and a decline observed at 30 minutes (see FIGS. 14C and 14D). Lastly, whether β-arrestin is required for these compound effects was determined by measuring ERK1/2 phosphorylation following stimulation in βarr1/2 siRNA-treated cells. Treatment with Cmpd-30 resulted in significant ERK1/2 activation in control siRNA-transfected cells. In contrast, knockdown of βarr1/2 markedly impaired the extent of Cmpd30-induced ERK1/2 phosphorylation (see FIGS. 14C and 14D).

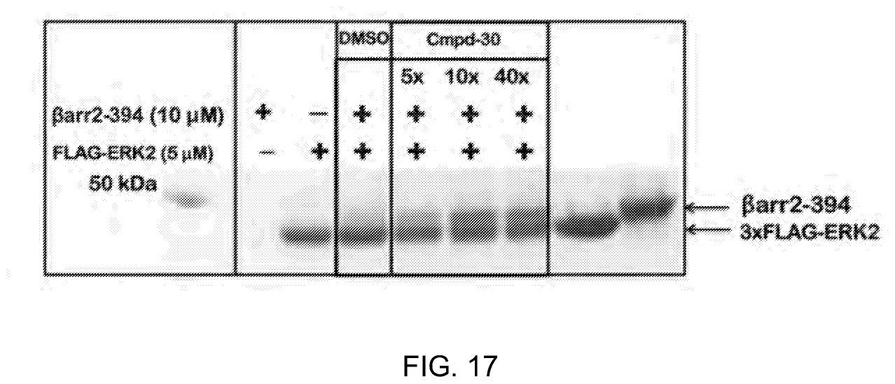

Given this surprising result on the ability of Cmpd-30 to activate ERK βarr1/2-dependent but receptor independent manner, next it was determined whether βarr2 and ERK2 can physically interact in the presence of Cmpd-30 by a pull-down assay. Purified 3×Flag-ERK2 was incubated with βarr2 in the presence of a series of concentrations of Cmpd-30 or vehicle. It was found that ERK2 forms a complex in vitro with βarr2 in a Cmpd-30-dependent manner (see FIG. 17). Altogether, the results clearly show that in the presence of Cmpd-30, βarr is capable of activating ERK1/2 in a receptor-independent manner. Together, these data support the conclusion that Cmpd-30 induces a unique βarr1/2 conformation that is suitable to bind to and scaffold unique cytosolic signaling units involving ERK1/2 in a receptor independent manner.

### Cmpd-30 Impairs Chemotaxis in Murine T-Cells

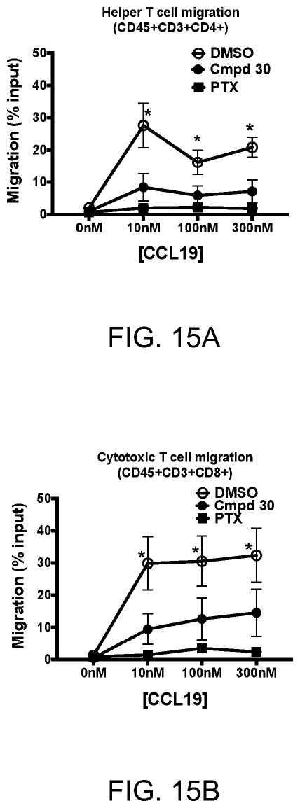

β-arrestins scaffold multiple proteins that control cell polarity and influence cellular migration downstream of GPCRs, including the chemokine receptors (Smith, J. S. et al. C-X-C Motif Chemokine Receptor 3 Splice Variants Differentially Activate Beta-Arrestins to Regulate Downstream Signaling Pathways. Mol Pharmacol 92, 136-150, doi:10.1124/mol.117.108522 (2017); Hunton, D. L. et al. Beta-arrestin 2-dependent angiotensin II type 1A receptor-mediated pathway of chemotaxis. Mol Pharmacol 67, 1229-1236, doi:10.1124/mol.104.006270 (2005); and Zoudilova, M. et al. beta-Arrestins scaffold cofilin with chronophin to direct localized actin filament severing and membrane protrusions downstream of protease-activated receptor-2. J Biol Chem 285, 14318-14329, doi:10.1074/jbc.M109.055806 (2010)). Given the surprising result on the ability of Cmpd-30 to activate ERK βarr1/2-dependent but receptor independent manner, whether compound 30 influences this complex receptor related β-arrestin function, T cell chemotaxis, was tested next. Murine peripheral node lymphocytes were isolated from wild type mice (leukocytes; T cells as well as CD4- and CD8-positive T cell subpopulations) and T cell chemotaxis was induced using chemoattractant CCL19, a ligand for the chemokine receptor 7 (CCR7). As shown in FIGS. 15A and 15B, pertussis toxin (PTX) completely inhibited the chemotactic response of T cells to CCL19. Consistent with its ability to inhibit βarr activity, Cmpd-30 was found to significantly impair CCL19-induced chemotaxis, of wild-type mouse total T cells as well as those of subpopulations CD4 and CD4+T cells (see FIGS. 15A and 15B). This effect of compound 30 on cell migration shows that the compound is inhibiting all receptor related barr functions including migrations of cells but activates receptor independent downstream signaling such as ERK MAP kinases activities.

Formation of Signaling-Competent, Homo-Oligomeric β-arr2 Structures by Cmpd-30

The potential mechanism by which Cmpd-30 regulates β-arrestin activity was examined next. It was hypothesized that Cmpd-30 may stabilize unique conformations of β-arrestin that is incapable of binding to an activated receptor but is able to bind to and activate signaling partners (e.g., ERK). To test this hypothesis, the structural organization and size distribution of the βarr2-Cmpd-30 complex by dynamic light scattering (DLS) was first examined (FIG. 16A). DLS of control βarr2 showed mono-disperse distribution, with an average hydrodynamic (particle size) diameter of 7.1 nm, corresponding to the monomeric state. Cmpd-30 significantly enhanced the particle diameter of βarr2 to 8.2 nm (P<0.01, respectively, vs. vehicle control treated; one-way ANOVA), a size that corresponds predominantly to a homo-dimeric state and, to a lesser extent, to a homo-trimeric state (FIG. 16A).

Negative-stain electron microscopy (EM) and single-particle 2D averaging analysis of the βarr2-Cmpd-30 complex was performed next. Reference-free 2D class averages were generated for all datasets. Class averages of control βarr2 samples showed predominantly a monomeric state with an overall dimension of ˜72 Å. The detail in the βarr2 alone control sample class averages revealed two distinct electron-dense lobes representing the N- and C-domains. These are notable structural features of βarr2 molecules and serve as a suitable reference to identify individual βarr2 particles (see FIG. 16B). Class averages of βarr2-Cmpd-30 samples revealed structurally defined homo-oligomers, primarily dimers and trimers (particle average width ranging in 80-90 Å, coherent with the DLS analysis; FIG. 16B). Only a negligible portion of the control βarr2 samples formed lower order homo-oligomers. As directly observed in the 2D class averages, such homo-dimers/trimers of βarr2 in presence of Cmpd-30 appears to use the N- and C-domain lobes as site of attachment between βarr2 protomers. The dimers appear to consist of four distinct elongated densities, corresponding to four lobes. Trimers appear to use two domains as the site of interaction, have relatively larger and compact density, and appear slightly asymmetrically organized (associated with negative stain EM). The overall modulatory role of Cmpd-30 to drive the homo-dimer/trimer state of βarr2 in this study closely resembles the previously reported homo-trimer of βarr2 observed in presence of IP6. Taken together, these results confirm that Cmpd-30 promotes unique conformational organizations of β-arrestins, as homo-dimer and -trimer states, by ratcheting individual active βarr protomers together through interactions with N- and C-domain lobes. Such homo-oligomeric structural organizations of β-arrestins allow them to provide a unique signaling module that mediates signaling independent of agonist-activated receptors.

### A. Compositions

Structures for activator and inhibitor compounds are shown in FIGS. 7A and 7B, herein. Corresponding IUPAC names and PubChem CID numbers for activator compounds and inhibitor compounds are provided in Table 2 and Table 3, respectively, below:

TABLE 2
Activator Compounds

| ID | IUPAC Name | PubChem CID: |
|---|---|---|
| C6 | 6-[7-[5-ethyl-5-(5-ethyl-5-hydroxy-6-methyloxan-2-yl)-3- | 3888 |
| | methyloxolan-2-yl]-4-hydroxy-3,5-dimethyl-6-oxononyl]-2- | |
| | hydroxy-3-methylbenzoic acid | |
| C31 | (3,5-dibromo-4-hydroxyphenyl)-(2-ethyl-1-benzofuran-3-yl)methanone | 2333 |
| C32 | 21-methoxy-17,17-dimethyl-5-(3-methylbut-2-enyl)-3,12,16- | 317611 |
| | trioxapentacyclo[11.8.0.02,10.04,9.015,20]henicosa- | |
| | 1(13),4(9),5,7,14,18,20-heptaen-6-ol | |
| C33 | 4-[[(R)-(8-hydroxyquinolin-7-yl)-phenylmethyl]amino]benzoic acid | 3246000 |

TABLE 3
Inhibitor Compounds

| ID | IUPAC Name | PubChem CID: |
|---|---|---|
| C1 | (7Z)-4,8-dimethyl-12-methylidene-3,14- | 5353864 |
| | dioxatricyclo[9.3.0.02,4]tetradec-7-en-13-one | |
| C5 | (1R,2S,10S,11R,15S,18R)-9,10,15,18-tetrahydroxy-12,12- | 317640 |
| | dimethyl-6-methylidene-17- | |
| | oxapentacyclo[7.6.2.15,8.01,11.02,8]octadecan-7-one | |
| C18 | (3Z,5E)-3,5-bis[(3,4-dichlorophenyl)methylidene]-1-(3- | 5351376 |
| | morpholin-4-ylpropanoyl)piperidin-4-one;hydrochloride | |
| C26 | 2-[(E)-1-[4-(4- | 5351213 |
| | acetylphenoxy)phenyl]ethylideneamino]guanidine;nitrate | |
| C29 | 1-[2-[(6,7-dimethoxyisoquinolin-1-yl)methyl]-4,5- | 72351 |
| | dimethoxyphenyl]ethanone | |
| C30 | 5-(furan-2-ylmethylimino)-N,N-dimethyl-1,2,4-dithiazol-3- | 360474 |
| | amine;hydrobromide | |
| C38 | 1-[4-[4-(2,4-dichlorophenyl)butyl]phenyl]-6,6-dimethyl-1,3,5- | 308871 |
| | triazine-2,4-diamine | |
| C41 | 2-[(3,4-dimethoxyphenyl)methyl]-1,3-benzothiazole | 365132 |
| C42 | 2-[(E)-2-nitroethenyl]-1H-indole | 5382764 |
| C46 | (2-hydroxy-5,5,9-trimethyl-14-methylidene-10,15-dioxo-6- | 327536 |
| | tricyclo[11.2.1.04,9]hexadec-1(16)-enyl) acetate | |
| C64 | [(8Z)-3,8-dimethyl-12-methylidene-13-oxo-4,14- | 5860420 |
| | dioxatricyclo[9.3.0.03,5]tetradec-8-en-10-yl] acetate | |
| C74 | (6Z)-6-[(2-methoxyphenyl)methylidene]-3-(3-nitrophenyl)- | 5847653 |
| | [1,3]thiazolo[2,3-b][1,3]thiazol-4-ium-5-one | |

Accordingly, one aspect of the present disclosure provides a compound comprising, consisting of, or consisting essentially of the general formula (I) (termed Cmpd 30; ((Z)-3-((furan-2-ylmethyl)imino)-N,N-dimethyl-3H-1,2,4-dithiazol-5-amine)):

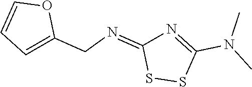

or a pharmaceutically acceptable salt, solvate, hydrate, prodrug, or derivative thereof.

Another aspect of the present disclosure provides a compound comprising, consisting of, or consisting essentially of the general formula (II) (termed Cmpd B29; (1-(2-((6,7-dimethoxyisoquinolin-1-yl)methyl)-4,5-dimethoxyphenyl)ethan-1-one)):

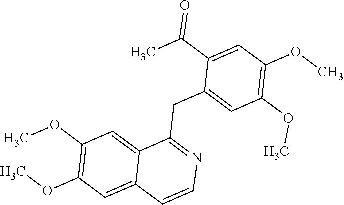

or a pharmaceutically acceptable salt, solvate, hydrate, prodrug, or derivative thereof.

Another aspect of the present disclosure provides a compound comprising, consisting of, or consisting essentially of the general formula (III) (termed Cmpd 31; ((3,5-dibromo-4-hydroxyphenyl)(2-ethylbenzofuran-3-yl)methanone)):

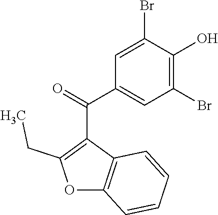

or a pharmaceutically acceptable salt, solvate, hydrate, prodrug, or derivative thereof.

Another aspect of the present disclosure provides a compound comprising, consisting of, or consisting essentially of the general formula (IV) (termed Cmpd 32; (4-(((8-hydroxyquinolin-7-yl)(phenyl)methyl)amino)benzoic acid)):

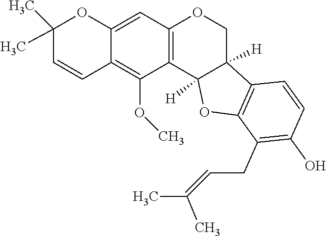

or a pharmaceutically acceptable salt, solvate, hydrate, prodrug, or derivative thereof.

Additional aspects of the present disclosure provide compounds comprising, consisting of, or consisting essentially of at least one of the general formulae as disclosed herein in FIG. 7A or 7B, and having correspondingly designated IUPAC names as provided in Tables 2 or 3, or a pharmaceutically acceptable salt, solvate, hydrate, prodrug, or derivative thereof.

### B. Pharmaceutical Compositions

In another aspect, the present disclosure provides compositions comprising one or more of compounds as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### C. Methods of Use and Treatment

The compounds, salts, solvates, hydrates, prodrugs, and derivatives thereof as well as any pharmaceutical compositions thereof as described herein have many potential applications, such as modulating (e.g., decreasing or inhibiting) βarrs activity.

Accordingly, another aspect of the present disclosure provides a method of modulating βarr activity in a cell comprising, consisting of, or consisting essentially of administering to the cell an effective amount of a compound as provided herein such that the βarr activity if modulated.

By “modulate” is meant to alter (e.g., increase or decrease) and refers to the ability of a compound to increase or decrease the activity, function, and/or expression of βarrs, where βarr activity or function may include the GPCR activity of said βarrs. Modulation may be assessed either in vitro or in vivo. Modulation, as described herein, includes the inhibition or activation of βarr activity, function, and/or the downregulation or upregulation of βarr expression, either directly or indirectly.

Another aspect of the present disclosure provides a method of inhibiting βarr activity in a cell and/or subject, the method comprising, consisting of, or consisting essentially of administering to the cell and/or subject an effective amount of a compound selected from the group consisting of Cmpd 30, Cmpd B29 and combinations thereof such that the βarr activity is inhibited in the cell and/or subject.

Another aspect of the present disclosure provides a method of activating βarr activity in a cell and/or subject, the method comprising, consisting of, or consisting essentially of administering to the cell and/or subject an effective amount of a compound selected from the group consisting of Cmpd 31, Cmpd 32 and combinations thereof such that the βarr activity is activated in the cell and/or subject.

The compounds according to the present disclosure may be administered to the cells on an in vivo basis (e.g., contact with cells takes place within the body of a subject) or ex vivo (e.g., contact with the cells takes place in an in vitro setting after being removed from the subject and are then reintroduced to a subject after treatment, in accordance with procedures which are most typically employed). Also, within the scope of the present disclosure is the use of the compound provided herein for research purposes, where cell lines maintained in a laboratory setting are put in contact with said compounds in an in vitro setting.

Another aspect of the present disclosure provides a method of treating a βarr-associated disease in a subject, the method comprising, consisting of, or consisting essentially of administering to the subject a therapeutically effective amount of a compound as provided herein such that the βarr-associated disease is treated in the subject.

In some embodiments, the βarr-associated disease is selected from the group consisting of cancer, asthma, metabolic diseases, chronic pain, cardiovascular diseases, neurological diseases, and combinations thereof.

Another aspect of the present disclosure provides a method of inhibiting chemotaxis of T cells in a subject, the method comprising, consisting of, or consisting essentially of administering to the subject a therapeutically effective amount of a compound as provided herein such that the chemotaxis of T cells in the subject is inhibited. In some embodiments, the compound comprises Cmpd 30.

### D. Kits

The present disclosure further provides kits for modulating βarr in a subject and/or treating a βarr-associated disease and/or condition in a subject, the kit comprising, consisting of, or consisting essentially of a compound as provided herein, pharmaceutical carrier(s), and instructions for using the kit components.

Yet another aspect of the present disclosure provides all that is disclosed and illustrated herein.

The following Examples are provided by way of illustration and not by way of limitation.

### EXAMPLES

### Cell Culture

HEK293 and U2OS cell lines were cultured in minimum Eagle's media (MEM) supplemented with 2 mM 1-glutamine, penicillin-streptomycin, and 10% fetal bovine serum, and maintained in an incubator with 5% CO₂ at 37° C. U2OS cell lines used for β-arrestin recruitment or receptor internalization assays were cultured as described by the manufacturer (DiscoveRx, Fremont, Calif.). For chemotaxis assays, mouse leukocytes were prepared as those previously described (Smith, J. S. et al. C-X-C Motif Chemokine Receptor 3 Splice Variants Differentially Activate Beta-Arrestins to Regulate Downstream Signaling Pathways. Mol Pharmacol 92, 136-150, doi:10.1124/mol.117.108522 (2017)). Briefly, leukocytes were obtained by passing cells isolated from the spleen and subjected to erythrocyte lysis through a 70-μm filter, were suspended in RPMI 1640 medium containing 5% FBS before using them for experiment. Transient transfections were performed using FuGENE 6 (Promega; Madison, Wis.) according to the manufacturer's instructions.

Small Interfering RNA (siRNA) Silencing of β-Arrestin Expression

Knockdown of the expression levels of β-arrestin were performed as described and functionally validated previously (Wang, J. et al. Galphai is required for carvedilol-induced beta1 adrenergic receptor beta-arrestin biased signaling. Nat Commun 8, 1706, doi:10.1038/s41467-017-01855-z (2017)). A non-silencing siRNA duplex sequence was also used as negative control (see Wang, et al.) HEK293 cells stably expressing FLAG-tagged β2AR were seeded into 10 cm dish on the day before to reach 40-50% confluence at the time of transfection. SiRNA were transfected using GeneSilencer Transfection Reagent (Genlantis) according to the manufacturer's protocol. In brief, 20 μg siRNA and 240 μl siRNA dilution buffer were added into 180 μL serum-free medium, whereas 51 μL of transfection reagent was mixed with 300 μL serum-free medium. Both solutions were allowed to stand for 5 min at room temperature, then combined and incubated for additional 20 min. The mixture was then added to cells in the 100 mm dish with 4 mL serum-free medium. After 4 h incubation at 37° C. and 5% CO₂, 5.5 ml of MEM containing 20% FBS and 2% penicillin-streptomycin were added into the dish. About sixty hours later, the cells were split and seeded into 6- or 12-well dishes for ERK activation assay.

### Compound Library and Reagents

A collection of structurally diverse, drug-like small-molecule compound library (DDLC) screened in this work was obtained from the NCI/DTP Open Chemical Repository. The compound library is comprised of ˜3.5K compounds that structurally represent over 250K unique small molecules. The majority of compounds were >95% pure as certified by the supplier (NCI DTP Discovery Services). Powdered compounds were dissolved in 100% dimethyl sulfoxide (DMSO; Thermo Fisher Scientific) and stored at −20° C. Carvedilol, isoproterenol and angiotensin II were purchased from Sigma-Aldrich. [Arg8]-Vasopressin (AVP) was purchased from GenScript. Isoproterenol, carvedilol and β-arrestin small-molecule modulators were dissolved in DMSO and stored at −20° C. Rat βarr1 and 2 (and similarly their truncated forms, at residue 393 and 394); and Fab30 were purified as previously described (Shukla, A. K. et al. Structure of active beta-arrestin-1 bound to a G-protein-coupled receptor phosphopeptide. Nature 497, 137-141, doi:10.1038/nature12120 (2013)). Protein concentrations for each protein were determined by ultraviolet absorption at 280 nm and extinction coefficients estimated using the ExPASy ProtParam tool.

### High-Throughput Screening (HTS) Differential Scanning Fluorimetry (DSF)-Based Thermal Shift Assay (TSA)

A high-throughput differential scanning fluorimetry-based thermal shift screen was developed to identify potential βarr small-molecule modulators present in the collection of structurally diverse, drug-like small-molecule compound library described above. The screen was performed using the StepOnePlus™ Real-Time PCR System (Applied Biosystems, Foster City, Calif.). Melting temperature changes were monitored with the use of a reporter fluorescent probe, SYPRO Orange (Thermo Fisher Scientific). Proteins were buffered in a 20 mM HEPES pH 7.5, 100 mM NaCl solution. All reactions were set up in a 96-well plate at final volumes of 20 μl. Small-molecule test compounds were plated from DMSO stock solutions into 96 well plates (1 compound/well) at a final compound concentration of 100 μM. The final DMSO concentration per well was 1%. βarr1 or βarr2 final concentration was maintained at 5 μM per well. Vehicle (DMSO), negative, and positive controls were also included in each plate. Negative controls contained SYPRO Orange in buffer by itself (no βarr protein). Three known βarr binders were individually tested under described assay conditions: ‘V2Rpp’, a phosphorylated peptide corresponding to the C-terminus of the vasopressin-2 receptor (V2R); Fab30, an antigen-binding fragment that recognizes V2Rpp bound active βarr conformation; and inositol hexakisphosphate (IP6). Amount of Fab30 or IP6 was used. Based on the responses, V2Rpp was selected as a positive control and present in each plate during screening. Excitation and emission filters for the SYPRO-Orange dye were set to 475 nm and 580 nm, respectively. The temperature was raised with a step of 0.5° C. per 30 second from 25° C. to 99° C. and fluorescence readings were taken at each interval. Melting curves were directly exported from the instrument and were analyzed using Applied Biosystems® Protein Thermal Shift™ Software. Melting temperature (Tm) was calculated by plotting the first derivative of the fluorescence emission as a function of temperature [−dF/dT]. The difference between the Tm of the protein-compound complex and the Tm of the apo protein represented the thermal shift (ΔTm), which is a measure of ligand binding to a protein of interest. All final Tm measurements for the best hits were carried out three times. Saturation binding measurements to estimate the binding affinity of hit compounds were performed in 96 well plates in presence of a 5 μM of βarr1 or βarr2, and at increasing concentrations of compound.

### Measurement of β-Arrestin Recruitment

β-arrestin recruitment to the agonist-activated receptor (02V2R) was measured using the DiscoveRx PathHunter β-arrestin assay (Ahn, S. et al. Allosteric “beta-blocker” isolated from a DNA-encoded small molecule library. Proc Natl Acad Sci U SA 114, 1708-1713, doi:10.1073/pnas.1620645114 (2017)). Briefly, the assay uses enzyme fragment complementation, where the receptor is fused to an inactive portion of the β-gal enzyme (ProLink™ tag), and co-expressed in U2OS cells stably expressing βarr2 fused to the complementary portion of β-gal. Agonist-stimulated recruitment of βarr2 to the receptor results in the formation of functionally active β-gal enzyme. Addition of substrate generates a chemiluminescence signal directly correlated to the extent of recruitment. U2OS cells co-expressing β2V2R and βarr2 were plated in 96-well clear-bottomed plates at a density of 25,000 cells per well (80 μL per well), 24 hours prior to compound treatment. On the day of experiment, cells were first treated with compound or vehicle for 30 min, followed by agonist (Iso) stimulation for 60 min at 37° C. Cells were then treated with PathHunter™ detection reagents for 90 min at 37° C., and luminescence signals were measured using a CLARIOstar microplate reader (BMG Labtech Inc., Cary, N.C.).

### Measurement of Receptor Internalization Using DiscoverRx

β-arrestin-mediated activated receptor internalization was measure using the DiscoveRx PathHunter activated receptor endocytosis assay according to the manufacturer's protocols (DiscoverRx, Fremont, Calif.). Briefly, an Enzyme Acceptor (EA)-tagged βarr and a ProLink tag localized to endosomes are stably expressed in U2OS cells. Untagged β2V2R is transiently expressed (4 ug of DNA). The next day, cells (25,000 cells per well) were then seeded in white 96-well clear-bottomed assay plates and incubated for 24 h before experiments. On the day of experiment, cells were first treated with compound or vehicle for 30 min, followed by a series concentrations of agonist (Iso) stimulation for 60 min at 37° C. Receptor internalization was detected as luminescence resulting from the complementation of β-galactosidase fragments (Enzyme Acceptor and ProLink) within endosomes. Luminescence signals were measured on a CLARIOstar microplate reader using the PathHunter Detection kit (DiscoveRx).

### BRET Based Receptor Internalization Assay

BRET-based assays were performed to measure receptor internalization using two complementary bystander BRET-formats. First, the association of the receptor (β2V2R-RlucII or V2R-RlucII, BRET donor) with the early endosome (2×FYVE-mVenus, BRET acceptor) was directly measured following agonist stimulation as described previously (Smith, J. S. et al. C-X-C Motif Chemokine Receptor 3 Splice Variants Differentially Activate Beta-Arrestins to Regulate Downstream Signaling Pathways. Mol Pharmacol 92, 136-150, doi:10.1124/mol.117.108522 (2017)). Second, receptor internalization following agonist stimulation was determined by measuring BRET signal from labeled receptor (β2V2R-RlucII or V2R-RlucII, BRET donor) and plasma membrane component (myr-palm-mVenus, BRET acceptor). Cells were transfected with 2.5 μg V2R-RlucII and 10 μg 2×FYVE-mVenus or 2 μg of myr-palm-mVenus using FuGENE HD (Promega, Madison, Wis.) in an individual 10 cm dish. ˜24 hours post-transfection, cells were seeded in white 96-well clear-bottomed plates at a density of 75,000 cells per well. The following day cells were washed and the media was changed to Hanks' Balanced Salt Solution (HBSS) buffer supplemented with 10 mM HEPES for 1 hours at 37° C. Cells were pretreated with βarr modulating compounds (50 μM) or vehicle, then incubated for 30 min at 37° C. Cells were stimulated with increasing doses of agonist isoproterenol or arginine vasopressin (GenScript). Coelenterazine-h was added to the cells 5 min prior to BRET measurements to a final concentration of 5 μM. BRET measurements were performed using the Synergy2 (BioTek®) microplate reader with a filter set of 410/80 nm and 515/30 nm to detect the RlucII (donor) and mVenus (acceptor) light emissions, respectively. The BRET signal was determined by calculating the ratio of the light intensity emitted by the acceptor over the light intensity emitted by the donor. Net BRET was calculated using this ratio and subtracting the same ratio measured from cells expressing only the BRET donor.

### Isothermal Titration Calorimetry (ITC)

ITC measurements were made using the MicroCal iTC200 system (MicroCal, Malvern, Pa.). Purified βarr1 or βarr2 was dialyzed in 20 mM HEPES, pH 7.4, containing 150 mM NaCl (HN buffer). The dialysis buffer was used to dilute DMSO stock solutions of βarr small molecule modulators to the final concentration used for measurements. ITC experiments were performed by loading βarr1 or βarr2 at 30 μM into the sample cell and 300 μM βarr-modulator compound into the injection syringe. The system was equilibrated to 25° C. Titration curves were initiated by a 0.4 μL injection from syringe, followed by 2.0 μL injections (at 180 s intervals) into the sample cell containing βarr1 or βarr2. During the experiment, the reference power was set to 7 μcal·s−1 and the sample cell was stirred continuously at 750 rpm. Raw data, excluding the peak from the first injection, were baseline corrected, peak area integrated, and normalized. Data were analyzed using MicroCal Origin software program to obtain association constant (Ka=1/Kd), stoichiometry (N), and thermodynamic parameters such as enthalpy (ΔH) and entropy (ΔS) of binding.

### Radioligand Binding Experiments

To determine the influence of small-molecule βarr modulators on βarr-promoted high-affinity agonist binding receptor state in vitro, [³H]-methoxyfenoterol (³H-Fen) binding experiments were performed using phosphorylated β2V2R (pβ2V2R) in Sf9 native membranes. Membranes were prepared following the procedures described earlier Ahn, S. et al. Allosteric “beta-blocker” isolated from a DNA-encoded small molecule library. Proc Natl Acad Sci USA 114, 1708-1713, doi:10.1073/pnas.1620645114 (2017). The reaction mixture (100 μL) contained pβ2V2R Sf9 membranes, ³H-Fen (12.6 Ci/mmol at its K-high, 4.3 nM), βarr1/2 (2 μM) plus βarr small-molecule modulators at the indicated concentrations or vehicle. ³H-FEN binding with purified heterotrimeric Gs was performed using the same membranes in the G protein assay buffer (25 mM HEPES, pH 7.4, 100 mM NaCl, 2 mM EDTA, 12.5 mM MgCl₂). Nonspecific radioligand binding was determined in reactions that contained the antagonist propranolol (20 μM). Following a 90 min incubation at room temperature, binding assays were terminated by harvesting the reaction mixture onto PEI-soaked GF/B filters and washing three times with cold buffer (Brandel Harvester, Gaithersburg, Md.). Bound [3H] was extracted overnight with 5 mL of scintillation fluid, quantified, and expressed as specific binding.

### Cytotoxicity Assay

MTT assay was performed according to the manufacturer's instructions (Roche). HEK293 and U2OS cells were seeded in 96-well plates (5×10³ cells per well). The following day, cells were treated with or without increasing concentrations of compounds (5 μM to 250 μM) for 24 hours. To evaluate potential cytotoxic effects of the compounds, cells were incubated with MTT reagent (Roche) and cell lysis buffer (100 μL of 10% SDS in 0.01 M HCl or 10% SDS, 50% N,N-dimethylformamide, pH 4.7) for 4 hours at 37° with vigorous mixing. The optical density (OD) was measured at 595 nm (the absorbance of each sample was measured at 560 and 670 nm). Percent cell viability was calculated relative to vehicle-treated cells after background subtraction. 50% cell growth inhibitory concentrations (IC₅₀) were determined from the linear portion of the plotted curves using GraphPad Prism software.

### Chemotaxis Assays

Chemotaxis assays were conducted similarly to those previously described Smith, J. S. et al. C-X-C Motif Chemokine Receptor 3 Splice Variants Differentially Activate Beta-Arrestins to Regulate Downstream Signaling Pathways. Mol Pharmacol 92, 136-150, doi:10.1124/mol.117.108522 (2017)). Briefly, mouse leukocytes, obtained by passing cells isolated from the spleen and subjected to erythrocyte lysis through a 70-μm filter, were suspended in RPMI 1640 medium containing 5% FBS. For the assay, 1×10⁶ cells in 100 μl of medium were added to the top chamber of a 6.5 mm diameter, 5-μm pore polycarbonate Transwell insert (Costar). Subsequently, cells were treated with βarr small-molecule modulators or vehicle for 30 min, followed by increasing concentrations of CCL19 suspended in 600 μL of the medium in the bottom chamber for 2 hours at 37° C. T cells migrated to the bottom of chamber were recovered, resuspended, washed, stained with a Live/Dead marker (Aqua Dead, ThermoFisher) and antibodies to cell surface markers (CD3, CD4, CD8, and CD45), and fixed with paraformaldehyde. The number of migrated T cells was measured by flow cytometric analysis with a BD LSRII Flow cytometer. Flow cytometry was performed in the Duke Human Vaccine Institute Research Flow Cytometry Facility (Durham, N.C.). CountBright beads (ThermoFisher) were added immediately after bottom chamber resuspension to correct for differences in final volume and any sample loss during wash steps. A 1:10 dilution of input cells was similarly analyzed. Specific T cell migration was calculated by dividing the number of migrated cells by the number of input cells.

FRET-Based cAMP Accumulation Measurement Assay

To measure cellular cAMP production in live cells mediated by stimulatory G protein, Gαs-coupled β₂AR activation, FRET-based Epac sensors were used as described previously (Violin, J. D. et al. beta2-adrenergic receptor signaling and desensitization elucidated by quantitative modeling of real time cAMP dynamics. J Biol Chem 283, 2949-2961, doi:10.1074/jbc.M707009200 (2008)). The Epac2 (ICUE2) sensor contains a CFP and YFP FRET pair. HEK293 cells stably expressing the ICUE2 cells were plated in poly-D-lysine-coated black 96 well plates (Corning) at a cell density of 50,000 cells per well. 16 hours after plating, cells were PBS-washed, then incubated in HEPES-buffered saline solution (150 mM NaCl, 5 mM KCl, 1 mM MgCl2, 2 mM CaCl2, 10 mM glucose, and 10 mM HEPES, pH 7.4) for one hour. Cells were either treated with βarr small-molecule modulators or DMSO for 15 min and monitored for changes in baseline fluorescent activity. cAMP accumulation was initiated by isoproterenol injection and fluorescence was measured at 5 second intervals for 15 min. cAMP accumulation results in a decrease in the FRET signal intensity (CFP excitation at 405 nm and YFP emission at 530 nm) and this was quantified as the integrated change in the FRET ratio (CFP/YFP).

### Intracellular Calcium Measurement

Intracellular [Ca²⁺] release was measured using FLIPR Calcium 6 with the FlexStation 3 microplate reader according to the manufacturer's instructions (Molecular Devices, LLC). Briefly, HEK293 cells stably expressing human AT1aR were seeded in poly-D-lysine-coated black 96-well assay plates (35,000 cells per well) and incubated for 24 hours. On the day of experiment, cell plates were loaded with the appropriate calcium kit reagents and then treated with βarr small molecule modulators or vehicle for 30 min. Basal fluorescence (F_{O}) was measured, agonist was applied while fluorescence (F) intensity was monitored in real time.

### Live Cell ERK1/2 Activation Assay

To assess the effect(s) of βarr small-molecule modulators on receptor-stimulated β-arr dependent ERK1/2 activation, HEK293 cells stably expressing β₂AR plates were starved for 6 hours in serum-free medium prior to stimulation (Wisler, J. W. et al. A unique mechanism of beta-blocker action: carvedilol stimulates beta-arrestin signaling. Proc Natl Acad Sci USA 104, 16657-16662, doi:10.1073/pnas.0707936104 (2007); Wang, J. et al. Galphai is required for carvedilol-induced beta1 adrenergic receptor beta-arrestin biased signaling. Nat Commun 8, 1706, doi:10.1038/s41467-017-01855-z (2017)). Cells were pretreated with βarr small-molecule modulators or vehicle, then incubated for 30 min at 37° C. After stimulation with appropriate receptor ligand (carvedilol or isoproterenol) for indicated time points, reactions were terminated by the addition of Laemmli sample buffer. The cell lysates were sonicated for 15 sec (2×) and clarified by centrifugation 14,000×g (4° C., 15 min). Proteins were resolved on an SDS-PAGE gel, transferred to nitrocellulose membranes, and immunoblotted using rabbit polyclonal anti-phospho-p44/42 MAPK (ERK1/2) antibody (1:2000; Cell Signaling), anti-MAPK1/2 (ERK1/2) antibody (1:10,000; Millipore-Sigma), anti-β-arrestin1 antibody (A1CT; 1:3000) or anti-β-arrestin2 antibody (A1CT; 1:3000, Lefkowitz lab, Duke University). Protein bands on the membrane were detected with SuperSignal West Pico enhanced chemiluminescent substrate (Thermo Fisher) and captured using a ChemiDoc-XRS charge-coupled device camera system (Bio-Rad Laboratories). Bands were quantified by densitometry using Image Lab (Bio-Rad, Hercules, Calif.) and GraphPad Prism software was used for data analyses.

For experiments involving siRNA transfection and knockdown βarrs, after stimulation cells were lysed in ice-cold 1% NP-40 lysis buffer [20 mM tris-HCl (pH 7.4), 137 mM NaCl, 20% glycerol, 1% NP-40, 2 mM sodium orthovanadate, 1 mM phenylmethylsulfonyl fluoride, 10 mM sodium fluoride, aprotinin (10 μg/ml), leupeptin (5 μg/ml), and phosphatase inhibitors (Phos-STOP; Roche)], rotated for 45 min, and cleared of insoluble debris by centrifugation at 14,000×g (4° C., 15 min). After which the supernatant was collected, protein concentration determined, and equal amounts of cell lysate were mixed with 2×Laemmli sample buffer. Samples were analyzed afterwards essentially as described above.

### Dynamic Light Scattering (DLS)

DLS was performed on a Zetasizer Nano ZS instrument (Malvern Instruments, UK) at an excitation He—Ne laser source of 633 nm and a detector at a scattering angle of 173°. The measurements were obtained at 25° C. and defined time intervals. A low volume (100 μl) disposable sample cuvette was used (BRAND, Wertheim, Germany). The protein solutions used for all particle size measurements were at a final βarr2 (truncated βarr2 at 394) concentration of ˜1 mg/mL (20 μM) in a buffer consisting of 20 M HEPES (pH 7.4) and 150 mM NaCl in presence of Cmpd-30 (60 μM), IP6 (300 uM) or vehicle (DMSO). Size distributions of scattering particles of different βarr2 sample were obtained after data analysis performed on intensity and volume size distribution curves and the molecular mass and Z-average size calculated using Malvern DTS software.

### Electron Microscopy (EM) Image Acquisition, Processing, Analysis, and 2D Image Classification

To prepare for EM visualization of the architecture and structural organization of βarr2-Cmpd30 complexes, purified βarr2 (394) was diluted to 0.5 μM and incubated with 5 μM Cmpd-30 or vehicle (DMSO) in HN buffer (20 mM HEPES pH 7.4 and 150 mM NaCl) for 7 min at room temperature followed by dilution 10× into HN buffer containing 1 μM Cmpd-30, before negative staining. The positive control, βarr2 bound to IP6 sample was prepared similarly (5 μM and 250 μM, respectively) and was diluted 100× into HN buffer containing 10 μM IP6. Grids were prepared using conventional negative-staining protocols as described previously (Peisley, A. & Skiniotis, G. 2D Projection Analysis of GPCR Complexes by Negative Stain Electron Microscopy. Methods Mol Biol 1335, 29-38, doi:10.1007/978-1-4939-2914-6_3 (2015). Briefly, 3 μL of samples were applied onto a glow-discharged EM carbon-coated copper grid, adsorbed on the grid surface, blotted, stained, and air dried before imaging. EM grids were examined with a FEI Tecnai G2 Twin electron microscope and operated at an acceleration voltage of 120 kV. Images were recorded with an Eagle 2K CCD camera at a magnification of ×65,200 and a defocus value of ˜1.5 μm. Two-dimensional EM reference-free alignment and classification of particle projections were performed with the iterative stable alignment and clustering procedure (ISAC)(Yang, Z., et al., Iterative stable alignment and clustering of 2D transmission electron microscope images. Structure 20, 237-247, doi:10.1016/j.str.2011.12.007 (2012)). EM single particles were both automatically and manually selected using Boxer (part of the EMAN 2.1 software suite)(Tang, G. et EMAN2: an extensible image processing suite for electron microscopy. J Struct Biol 157, 38-46, doi:10.1016/j.jsb.2006.05.009 (2007)). A total of ˜250,000 0° particles projections were picked representing all the three βarr2 samples (with vehicle, Cmpd-30 or IP6) and windowed into 96×96-pixel images (with the RELION 2.1-beta-1 software package) and subjected to ISAC. After subjecting to ten cycles of reference-free alignments, 150 classes were obtained, accounting for ˜177,000 particles (70% of the entire dataset). Based on the first classification, we observed a heterogenous sample particles composition, including a monomeric βarr2 that has two distinct electron-dense lobes representing the N- and C-domains; a second more compact monomeric βarr2 particles; and a third more dominant in Cmpd-30 and IP6 samples, consisting of lower order homo-oligomeric assembly of βarr2 (dimer and trimer states). Next, only the well resolved particles were selected, removing underrepresented and poorly resolved particles. The remaining particles were subjected to a second multi-reference alignment to improve classification. To determine the βarr2 architectures, each class average was designated as monomer or lower order homo-oligomeric state (dimer/trimer). Class averages of control βarr samples showed predominantly a monomeric state (97%; with an overall dimension of ˜72 Å) and only a minority of them were present as homo-oligomers. While class averages of Barr2 samples bound to Cmpd-30 and IP6 were represented almost exclusively as homo-oligomers (dimers/trimers) with 95% and 100%, respectively.

### Statistical Analysis.

Statistical analysis and curve fitting were done using Prism 6 (GraphPad Software). For statistical comparison, one-way analysis of variance (ANOVA) with p-values of <0.05 considered significant.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

We claim:

1. A compound comprising the general formula (I) (termed Cmpd 30): 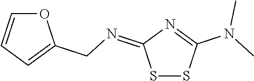
or a pharmaceutically acceptable salt, solvate, hydrate, prodrug, or derivative thereof.

2. A compound comprising the general formula (II) (termed Cmpd B29): 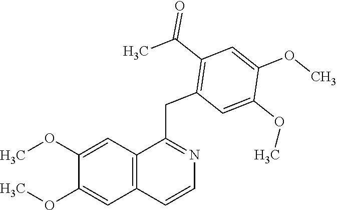
or a pharmaceutically acceptable salt, solvate, hydrate, prodrug, or derivative thereof.

3. A compound comprising the general formula (III) (termed Cmpd 31): 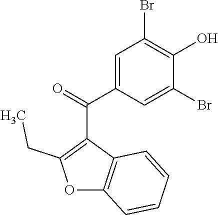
or a pharmaceutically acceptable salt, solvate, hydrate, prodrug, or derivative thereof.

4. A compound comprising the general formula (IV) (termed Cmpd 32): 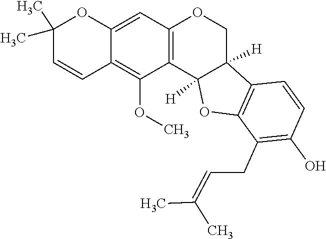
or a pharmaceutically acceptable salt, solvate, hydrate, prodrug, or derivative thereof.

5. A pharmaceutical composition comprising a compound as in any of the preceding claims and a pharmaceutically acceptable carrier and/or excipient.

6. A method of modulating β-arrestin (βarr) activity in a cell comprising administering to the cell an effective amount of a compound as in any of the preceding claims such that the β-arrestin (βarr) activity is modulated in the cell.

7. A method of modulating β-arrestin (βarr) activity in a subject comprising administering to the subject an effective amount of a compound as in any of claim 1-4 or 5 such that the β-arrestin (βarr) activity is modulated in the subject.

8. A method of inhibiting βarr activity in a cell, the method comprising administering to the cell an effective amount of a compound selected from the group consisting of Cmpd 30, Cmpd B29 and combinations thereof such that the βarr activity is inhibited in the cell.

9. A method of inhibiting βarr activity in a subject, the method comprising administering to the subject an effective amount of a compound selected from the group consisting of Cmpd 30, Cmpd B29 and combinations thereof such that the βarr activity is inhibited in the subject.

10. A method of activating βarr activity in a cell, the method comprising administering to the cell an effective amount of a compound selected from the group consisting of Cmpd 31, Cmpd 32 and combinations thereof such that the βarr activity is activated in the cell.

11. A method of activating βarr activity in a subject, the method comprising administering to the subject an effective amount of a compound selected from the group consisting of Cmpd 31, Cmpd 32 and combinations thereof such that the βarr activity is activated in the subject.

12. A method of treating a βarr-associated disease in a subject, the method comprising administering to the subject a therapeutically effective amount of a compound as provided herein such that the βarr-associated disease is treated in the subject.

13. The method according to claim 12 in which the βarr-associated disease is selected from the group consisting of cancer, asthma, metabolic diseases, chronic pain, cardiovascular diseases, neurological diseases, and combinations thereof.

14. A method of inhibiting chemotaxis of T cells in a subject, the method comprising administering to the subject a therapeutically effective amount of a compound as provided herein such that the chemotaxis of T cells in the subject is inhibited.

15. The method according to claim 14 in which the compound comprises Cmpd 30.

16. All that is described and illustrated herein.
